(19) United States
(12) Patent Application Publication
(10) Pub. No.: US 2019/0336005 A1
(43) Pub. Date: Nov. 7, 2019

(54) NON-INVASIVE FREQUENCY DOMAIN OPTICAL SPECTROSCOPY FOR NEURAL DECODING

(71) Applicant: HI LLC, Los Angeles, CA (US)

(72) Inventors: Jamu Alford, Simi Valley, CA (US); Roarke Horstmeyer, Durham, NC (US); Adam Marblestone, Arlington, MA (US)

(73) Assignee: HI LLC, Los Angeles, CA (US)

(21) Appl. No.: 16/379,090

(22) Filed: Apr. 9, 2019

Related U.S. Application Data
(60) Provisional application No. 62/666,926, filed on May 4, 2018.
(60) Provisional application No. 62/692,074, filed on Jun. 29, 2018.

(51) Int. Cl. A61B 5/00

(52) U.S. Cl. CPC A61B 5/0082; A61B 5/4064; A61B 5/0042

(57) ABSTRACT

An optical measurement system comprises an optical source assembly configured for intensity modulating sample light at multiple frequencies within a frequency range, and delivering the intensity modulated sample light along an optical path of an anatomical structure during a single measurement period, such that the intensity modulated sample light is scattered by the anatomical structure, resulting in signal light that exits the anatomical structure. The optical measurement system further comprises an optical detection assembly configured for detecting the signal light over the frequency range within the measurement period. The optical measurement system further comprises a processor configured for analyzing the detected signal light, and, based on this analysis, determining an occurrence and spatial depth of a physiological event in the anatomical structure.

### RELATED APPLICATION DATA

Pursuant to 35 U.S.C. § 119(e), this application claims the benefit of U.S. Provisional Patent Application 62/666,926, filed May 4, 2018, and U.S. Provisional Patent Application 62/692,074, filed Jun. 29, 2018, which are expressly incorporated herein by reference.

### FIELD OF THE INVENTION

The present inventions relate to methods and systems for non-invasive measurements in the human body, and in particular, methods and systems related to detecting physiological events in the human body, animal body, and/or biological tissue.

### BACKGROUND OF THE INVENTION

Measuring neural activity in the brain is useful for medical diagnostics, neuromodulation therapies, neuroengineering, or brain-computer interfacing. Conventional methods for measuring neural activity in the brain include diffusive optical imaging techniques, which employ moderate amounts of near-infrared or visible light radiation, thus being comparatively safe and gentle for a biological subject in comparison to X-Ray Computed Tomography (CT) scans, positron emission tomography (PET), or other methods that use higher-energy and potentially harmful radiation. Moreover, in contrast to other methods, such as functional magnetic resonance imaging (fMRI), these optically-based imaging methods do not require large magnets or magnetic shielding, and thus, can be scaled to wearable or portable form factors, which is especially important in applications, such as brain-computer interfacing.

There is an increasing interest in measuring fast-optical signals, which refers to changes in optical scattering that occur when light propagating through active neural tissue (e.g., active brain tissue) is perturbed through a variety of mechanisms, including, but not limited to, cell swelling, cell volume change, changes in membrane potential, changes in membrane geometry, ion redistribution, birefringence changes, etc. (see Hill D. K. and Keynes, R. D., “Opacity Changes in Stimulated Nerve,” J. Physiol., Vol. 108, pp. 278-281 (1949); Foust A. J. and Rector D. M., “Optically Teasing Apart Neural Swelling and Depolarization,” Neuroscience, Vol. 145, pp. 887-899 (2007)). Because fast-optical signals are associated with neuronal activity, rather than hemodynamic responses, fast-optical signals may be used to detect brain activity with relatively high temporal resolution.

However, because optical imaging techniques rely on light, which scatters many times inside brain, skull, dura, pia, and skin tissues, the light paths occurring in these techniques comprise random or “diffusive” walks, and therefore, only limited spatial resolution can be obtained by a conventional optical detector, often on the order of centimeters, with penetration depths being limited to a few millimeters. The reason for this limited spatial resolution is that the paths of photons striking the detector in such schemes are highly variable and difficult, and even impossible, to predict without detailed microscopic knowledge of the scattering characteristics of the brain volume of interest, which is typically unavailable in practice (i.e., in the setting of non-invasive measurements through skull for brain imaging and brain interfacing). In summary, light scattering has presented challenges for optical imaging techniques in achieving high spatial resolution deep inside tissue. Moreover, the diffusive nature of light propagation also creates challenges for measurements of fast changes in optical scattering inside tissue, since essentially all paths between source and detector are highly scattered to begin with.

Diffusive optical imaging techniques have been used to achieve nominal spatial resolution by locating a multitude of optical sources and detectors along the surface of the head that, despite the random propagation of light from the optical sources, can identify tube-like pathways through which photons are likely to travel during the random motion (see Gratton G., Fabiani M, “Fast-optical Imaging of Human Brain Function,” Frontiers in Human Neuroscience, Vol. 4, Article 52, pp. 1-9 (June 2010)). However, nearly all diffusive optical imaging techniques to date offer relatively poor temporal resolution (100 ms-1 sec per sample), as they are primarily designed to detect hemodynamics that vary on a similarly slow time scale.

Gratton, and others, have used a relatively simple frequency domain diffuse optical tomography (DOT) approach to measure fast-optical signals associated with neural activity by intensity modulating the light source at a specific modulation frequency (approximately 100 MHz) to sample the brain tissue. However, because this approach only samples the brain tissue at one modulation frequency, the detection sensitivity of fast-optical signals in the brain tissue is not maximized. Furthermore, this approach does not acquire spatial depth information of the fast-optical signals.

Another type of diffusive optical imaging technique, referred to as frequency-domain photon migration (FDPM), is used to measure the optical near-infrared (NIR) absorption and scattering properties of turbid media, which if living tissue, can provide quantitative functional biophysical information, such as deep tissue concentrations of chromophores (e.g., hemoglobin, water, and lipid) (see Thomas D. O'Sullivan, Keunsik No, Alex Matlock, Robert V. Warren, Brian Hill, Albert E. Cerussi, Bruce J. Tromberg, “Vertical-Cavity Surface-Emitting Laser Sources For Gigahertz-Bandwidth, Multiwavelength Frequency-Domain Photon Migration,” J. Biomed. Opt. 22 (10), 105001 (2017)). Although the FDPM technique described in O'Sullivan intensity modulates the light source at multiple frequencies, O'Sullivan discloses no means for measuring fast-optical signals within brain tissue using the FDPM technique, and furthermore, does not disclose any means for using the frequency information to obtain spatial depth information of any biologically inherent signals.

Still another type of diffusive optical imaging technique, referred to as interferometric Near-Infrared Spectroscopy (iNIRS) (see Borycki, Dawid, Kholiqov, Oybek, Chong, Shau Poh, Srinivasan, Vivek J., “Interferometric Near-Infrared Spectroscopy (iNIRS) for Determination of Optical and Dynamical Properties of Turbid Media,” Optics Express, Vol. 24, No. 1, Jan. 11, 2016), as well as swept source optical coherence tomography (SS-OCT), does obtain spatial depth information of a biological inherent signal. However, these techniques utilize holographic methods, mixing the detected light against a reference beam, thereby requiring a relatively complicated and expensive arrangement of components. Further, while the iNIRS or SS-OCT approaches are very sophisticated, they require the detection and measurement of speckles, presenting challenges in a highly attenuating medium, such as the human body, due to the very low number of photons that reach each detector. Thus, a very large number of detectors (or pixels) are required to individually detect the speckles, thereby further increasing the complexity and expense of the system. This complexity and expense will, of course, be magnified as the iNIRS system or SS-OCT system is scaled to increase the number of optical source-detector pairs for x-y (non-depth) spatial resolution.

There, thus, remains a need to provide a relatively simple non-invasive optical measurement system for measuring or detecting biologically inherent signals, such as fast-optical signals, in the brain at a sufficient spatial depth resolution, temporal resolution, and sensitivity.

### SUMMARY OF THE INVENTION

In accordance with one embodiment of the present inventions, an optical non-invasive measurement system comprises an optical source assembly configured for intensity modulating sample light at multiple frequencies within a frequency range (e.g., a frequency equal to or greater than 2 GHz, or even equal to or greater than 5 GHz, or in the frequency range of 1 GHz to 5 GHz, or even in the frequency range of 100 MHz to 10 GHz), and delivering the intensity modulated sample light along one or more optical paths in an anatomical structure (e.g., a brain) during a single measurement period, such that the intensity modulated sample light is scattered by the anatomical structure, resulting in signal light that exits the anatomical structure. The sample light may have a suitable wavelength, e.g., in the range of 350 nm to 1800 nm. The optical non-invasive measurement system may further comprise a controller configured for instructing the optical source assembly to sequentially intensity modulate sample light at the multiple frequencies over the frequency range within the measurement period, e.g., by sweeping the intensity modulation frequency of the intensity modulated sample light over the frequency range within the measurement period. Alternatively, the controller may be configured for instructing the optical source assembly to simultaneously intensity modulate sample light at the multiple frequencies.

In one embodiment, the optical source assembly comprises an electrical signal generator configured for outputting an electrical alternating current (AC) signal at the multiple frequencies, a first amplifier configured for amplifying the AC signal and outputting a drive signal, and an optical source (e.g., a vertical-cavity surface-emitting laser (VCSEL), a light emitting diode (LED), an edge emitting diode laser, or a flash lamp) configured for outputting the intensity modulated sample light at the multiple frequencies in accordance with the drive signal.

The optical non-invasive measurement system further comprises an optical detection assembly configured for detecting the signal light over the frequency range within the measurement period. In one embodiment, the optical detection assembly comprises an optical detector (e.g., a photodiode) configured for detecting the signal light and outputting an electrical physiological-encoded signal, a second amplifier configured for amplifying the physiological-encoded signal, and an analog-to-digital converter (ADC) configured for digitizing the amplified physiological-encoded signal into digital physiological-encoded data. The second amplifier may be, e.g., a lock-in amplifier configured for, in response to an electrical signal output by the optical source assembly at the multiple frequencies, amplifying the physiological-encoded signal comprises outputting an intensity and phase of the physiological-encoded signal, in which case, the ADC may be configured for digitizing the intensity and phase output by the lock-in amplifier into digital physiological-encoded data. The optical detector may comprise at least one discrete detector. Each of the discrete detector(s) may have an area greater than 30 μm², or even greater than 200 μm², but preferably less than 1000 μm².

The optical non-invasive measurement system further comprises a processor configured for analyzing the detected signal light, and, based on this analysis, determining an occurrence and spatial depth of a physiological event (e.g., a fast-optical signal) in the anatomical structure.

In one embodiment, the processor may be configured for analyzing the detected signal light in the frequency domain at one or more frequencies, and based on this analysis, determining the occurrence of the physiological event in the anatomical structure. For example, the processor may be configured for determining the occurrence of the physiological event in the anatomical structure by comparing a difference between the detected signal light to a baseline signal light (e.g., a user-specific model) at the one or more frequencies.

In another embodiment, the processor may be configured for analyzing the detected signal light in the time domain at one or more optical path lengths, and based on this analysis, determining the occurrence of the physiological event in the anatomical structure. For example, the processor may be configured for transforming a frequency domain representation of the detected signal light into a time domain representation (e.g., using an Inverse Fast Fourier Transform (IFFT)) of the detected signal light to obtain a measure of the detected signal light as a function of optical path length, in which case, the occurrence of the physiological event in the anatomical structure may be determined based on the measure of the detected signal light as a function of optical path length, e.g., by comparing a difference between the detected signal light to baseline signal light (e.g., a user-specific model) at the one or more optical path lengths.

In still another embodiment, the processor may be configured for analyzing the detected signal light in the time domain at one or more optical path lengths, and based on this analysis, determining the spatial depth of the physiological event in the anatomical structure. For example, the processor may be configured for transforming a frequency domain representation of the detected signal light into the time domain representation of the signal light (e.g., using an Inverse Fast Fourier Transform (IFFT)) to obtain a measure of the detected signal light as a function of optical path length. The spatial depth of the physiological event in the anatomical structure may be determined based on the measure of the detected signal light as a function of optical path length, in which case, the spatial depth of the physiological event in the anatomical structure may be determined based on the measure of the detected signal light as a function of optical path length, e.g., by comparing a difference between the detected signal light to baseline signal light (e.g., a user-specific model) at the one or more optical path lengths.

In yet another embodiment, the processor is configured for analyzing the detected signal light in the frequency domain at one or more frequencies, and based on this analysis, determining the spatial depth of the physiological event in the anatomical structure. For example, the processor may be configured for determining the spatial depth of the physiological event in the anatomical structure by comparing a difference between the detected signal light to a baseline signal light (e.g., a user-specific model) at the one or more frequencies.

The sample light may optionally have two different optical wavelengths (e.g., one equal to or greater than 850 nm, and another one in the range of 650 nm to 750 nm), in which case, the processor may be configured for analyzing the detected signal light, and, based on this analysis, determining an occurrence and spatial depth of the physiological event (e.g., a fast optical signal) in the anatomical structure at the first optical wavelength, and determining an occurrence and spatial depth of another physiological event (e.g., a blood oxygen concentration) in the anatomical structure at the second optical wavelength.

In accordance with a second aspect of the present inventions, an optical non-invasive measurement method comprises intensity modulating sample light at multiple frequencies within a frequency range (e.g., a frequency equal to or greater than 2 GHz, or even equal to or greater than 5 GHz, or in the frequency range of 1 GHz to 5 GHz, or even in the frequency range of 100 MHz to 10 GHz). The sample light may have a suitable wavelength, e.g., in the range of 350 nm to 1800 nm. In one method, the sample light is sequentially intensity modulated at the multiple frequencies by sweeping the intensity modulation frequency of the intensity modulated sample light over the frequency range within the measurement period. In another method, the sample light is simultaneously intensity modulated at the multiple frequencies. In still another method, intensity modulating the sample light at multiple frequencies within a frequency range comprises outputting an electrical alternating current (AC) signal at the multiple frequencies, amplifying the AC signal and outputting a drive signal, and outputting the intensity modulated sample light at the multiple frequencies in accordance with the drive signal. The intensity modulated sample light may be generating by one of, e.g., a vertical-cavity surface-emitting laser (VCSEL), a light emitting diode (LED), an edge emitting diode laser, and a flash lamp.

The method further comprises delivering the intensity modulated sample light along an optical path in an anatomical structure (e.g., a brain) during a single measurement period, such that the intensity modulated sample light is scattered by the anatomical structure, resulting in signal light that exits the anatomical structure, and detecting the signal light (e.g., using a photodiode) over the frequency range within the measurement period. In one method, detecting the intensity modulated signal light comprises detecting the signal light and outputting an electrical physiological-encoded signal, amplifying the physiological-encoded signal, and digitizing the amplified physiological-encoded signal into digital physiological-encoded data. Amplifying the physiological-encoded signal comprises outputting an intensity and phase of the physiological-encoded signal in response to an electrical signal output at the multiple frequencies, in which case, the intensity and phase is digitized into digital physiological-encoded data. The intensity modulated signal light may be detected with at least one discrete detector. Each of the discrete detector(s) may have an area greater than 30 μm², or even greater than 200 μm², but preferably less than 1000 μm².

The method further comprises analyzing the detected signal light, and determining an occurrence and spatial depth of a physiological event (e.g., a fast-optical signal) in the anatomical structure based on the analysis.

In one method, the detected signal light is analyzed in the frequency domain at one or more frequencies, and the occurrence of the physiological event in the anatomical structure is based on the analysis in the frequency domain. For example, the occurrence of the physiological event in the anatomical structure may be determined by comparing a difference between the detected signal light to a baseline signal light (e.g., a user-specific model) at the one or more frequencies.

In another method, the detected signal light is analyzed in the time domain at one or more optical path lengths, and the occurrence of the physiological event in the anatomical structure is determined based on the analysis in the time domain. For example, the frequency domain representation of the detected light can be transformed into a time domain representation (e.g., using an Inverse Fast Fourier Transform (IFFT)) to obtain intensity-optical path length information of the detected signal light, in which case, the occurrence of the physiological event in the anatomical structure may be determined based on intensity-optical path length information, e.g., by comparing a difference between the detected signal light to baseline signal light (e.g., a user-specific model) at the one or more optical path lengths.

In still another method, the detected signal light is analyzed in the time domain at one or more optical path lengths, and the spatial depth of the physiological event in the anatomical structure is determined based on the analysis in the time domain. For example, a frequency domain representation of the detected signal light can be transformed into a time domain representation of the detected signal light (e.g., using an Inverse Fast Fourier Transform (IFFT)) to obtain intensity-optical path length information of the detected signal light, wherein the spatial depth of the physiological event in the anatomical structure is determined based on intensity-optical path length information. The spatial depth of the physiological event in the anatomical structure may be determined based on the measure of the detected signal light as a function of optical path length, in which case, the spatial depth of the physiological event in the anatomical structure may be determined based on the measure of the detected signal light as a function of optical path length, e.g., by comparing a difference between the detected signal light to baseline signal light (e.g., a user-specific model) at the one or more optical path lengths.

In yet another method, the detected signal light is analyzed in the frequency domain at one or more frequencies, and the spatial depth of the physiological event in the anatomical structure is determined based on the analysis in the frequency domain. For example, the spatial depth of the physiological event in the anatomical structure is may be determined by comparing a difference between the detected signal light to baseline signal light (e.g., a user-specific model) at the one or more frequencies at the one or more frequencies.

The sample light may optionally have two different optical wavelengths (e.g., one equal to or greater than 850 nm, and another one in the range of 650 nm to 750 nm), in which case, the detected signal light may be analyzed, and, based on this analysis, an occurrence and spatial depth of the physiological event (e.g., a fast optical signal) in the anatomical structure may be determined at the first optical wavelength, and an occurrence and spatial depth of another physiological event (e.g., a blood oxygen concentration) in the anatomical structure may be determined at the second optical wavelength.

In accordance with a third aspect of the present inventions, an optical non-invasive measurement system comprises a plurality of paired optical source-detector combinations. Each of the paired optical source-detector combinations corresponds to a different optical path in an anatomical structure (e.g., a brain), and is configured for intensity modulating sample light at multiple frequencies within a frequency range (e.g., a frequency equal to or greater than 2 GHz, or even equal to or greater than 5 GHz, or in the frequency range of 1 GHz to 5 GHz, or even in the frequency range of 100 MHz to 10 GHz), and delivering the intensity modulated sample light along the respective optical path in the anatomical structure during a single measurement period, such that the intensity modulated sample light is scattered by the anatomical structure, resulting in signal light that exits the anatomical structure. The sample light may have a suitable wavelength, e.g., in the range of 350 nm to 1800 nm. Each of the paired optical source-detector combinations is further configured for detecting the respective signal light over the frequency range within the measurement period.

In one embodiment, the plurality of paired optical source-detector combinations comprises a single optical source assembly and multiple optical detection assemblies, such that a different optical path is created between the single optical source assembly and each respective optical detection assembly. In another embodiment, the plurality of paired optical source-detector combinations comprises multiple optical source assemblies and a single optical detection assembly, such that a different optical path is created between each respective optical source assembly and the single optical detection assembly. In still another embodiment, plurality of paired optical source-detector combinations comprises multiple optical source assemblies and multiple optical detection assemblies, such that different optical paths are created between each respective optical source assembly and each respective optical detection assembly.

The optical non-invasive measurement system may further comprise a controller configured for instructing each paired optical source-detector combination to sequentially intensity modulate sample light at the multiple frequencies over the frequency range within the measurement period, e.g., by sweeping the intensity modulation frequency of the intensity modulated sample light over the frequency range within the measurement period. Alternatively, the controller may be configured for instructing each paired optical source-detector combination to simultaneously intensity modulate sample light at the multiple frequencies.

In one embodiment, the paired optical source-detector combinations are created between at least one optical source assembly and at least one optical detector assembly. In this case, each of the optical source assembly(ies) may comprise an electrical signal generator configured for outputting an electrical alternating current (AC) signal at the multiple frequencies, a first amplifier configured for amplifying the AC signal and outputting a drive signal, and an optical source configured for outputting the intensity modulated sample light at the multiple frequencies in accordance with the drive signal. Each of the optical detection assembly(ies) may comprise an optical detector (e.g., a photodiode) configured for detecting the signal light and outputting an electrical physiological-encoded signal, a second amplifier configured for amplifying the physiological-encoded signal, and an analog-to-digital converter (ADC) configured for digitizing the amplified physiological-encoded signal into digital physiological-encoded data. The second amplifier may be, e.g., a lock-in amplifier configured for, in response to an electrical signal output by the optical source assembly at the multiple frequencies, amplifying the physiological-encoded signal comprises outputting an intensity and phase of the physiological-encoded signal, in which case, the ADC may be configured for digitizing the intensity and phase output by the lock-in amplifier into digital physiological-encoded data. The optical detector may comprise at least one discrete detector. Each of the discrete detector(s) may have an area greater than 30 μm², or even greater than 200 μm², but preferably less than 1000 μm².

The optical non-invasive measurement system further comprises a processor configured for analyzing the detected signal light for all of the paired optical source-detector combinations over the respective frequency ranges, and, based on this analysis, determining an occurrence and a location of a physiological event (e.g., a fast-optical signal) in at least two dimensions (which may include a spatial depth) in the anatomical structure.

In one embodiment, the processor may be configured for analyzing the detected signal light in the frequency domain at one or more frequencies, and based on this analysis, determining the occurrence of the physiological event in the anatomical structure. For example, the processor may be configured for determining the occurrence of the physiological event in the anatomical structure by comparing a difference between the detected signal light to a baseline signal light (e.g., a user-specific model) at the one or more frequencies.

In another embodiment, the processor may be configured for analyzing the detected signal light in the time domain at one or more optical path lengths, and based on this analysis, determining the occurrence of the physiological event in the anatomical structure. For example, the processor may be configured for transforming a frequency domain representation of the detected signal light into a time domain representation (e.g., using an Inverse Fast Fourier Transform (IFFT)) of the detected signal light to obtain a measure of the detected signal light as a function of optical path length, in which case, the occurrence of the physiological event in the anatomical structure may be determined based on the measure of the detected signal light as a function of optical path length, e.g., by comparing a difference between the detected signal light to baseline signal light (e.g., a user-specific model) at the one or more optical path lengths.

In still another embodiment, the processor may be configured for analyzing the detected signal light in the time domain at one or more optical path lengths, and based on this analysis, determining the spatial depth of the physiological event in the anatomical structure. For example, the processor may be configured for transforming a frequency domain representation of the detected signal light into the time domain representation of the signal light (e.g., using an Inverse Fast Fourier Transform (IFFT)) to obtain a measure of the detected signal light as a function of optical path length. The spatial depth of the physiological event in the anatomical structure may be determined based on the measure of the detected signal light as a function of optical path length, in which case, the spatial depth of the physiological event in the anatomical structure may be determined based on the measure of the detected signal light as a function of optical path length, e.g., by comparing a difference between the detected signal light to baseline signal light (e.g., a user-specific model) at the one or more optical path lengths.

In yet another embodiment, the processor is configured for analyzing the detected signal light in the frequency domain at one or more frequencies, and based on this analysis, determining the spatial depth of the physiological event in the anatomical structure. For example, the processor may be configured for determining the spatial depth of the physiological event in the anatomical structure by comparing a difference between the detected signal light to a baseline signal light (e.g., a user-specific model) at the one or more frequencies.

The sample light may optionally have two different optical wavelengths (e.g., one equal to or greater than 850 nm, and another one in the range of 650 nm to 750 nm), in which case, the processor may be configured for analyzing the detected signal light, and, based on this analysis, determining an occurrence and spatial depth of the physiological event (e.g., a fast optical signal) in the anatomical structure at the first optical wavelength, and determining an occurrence and spatial depth of another physiological event (e.g., a blood oxygen concentration) in the anatomical structure at the second optical wavelength.

In accordance with a fourth aspect of the present inventions, an optical non-invasive measurement method comprises defining a plurality of paired optical source-detector combinations, each of which corresponds to an optical path in an anatomical structure (e.g., a brain). The method further comprises intensity modulating sample light at multiple frequencies within a frequency range (e.g., a frequency equal to or greater than 2 GHz, or even equal to or greater than 5 GHz, or in the frequency range of 1 GHz to 5 GHz, or even in the frequency range of 100 MHz to 10 GHz) via each of the paired optical source-detector combinations. The sample light may have a suitable wavelength, e.g., in the range of 350 nm to 1800 nm. In one method, the sample light is sequentially intensity modulated at the multiple frequencies by sweeping the intensity modulation frequency of the intensity modulated sample light over the frequency range within the measurement period. In another method, the sample light is simultaneously intensity modulated at the multiple frequencies. In still another method, intensity modulating the sample light at multiple frequencies within a frequency range comprises outputting an electrical alternating current (AC) signal at the multiple frequencies, amplifying the AC signal and outputting a drive signal, and outputting the intensity modulated sample light at the multiple frequencies in accordance with the drive signal. The intensity modulated sample light may be generating by one of, e.g., a vertical-cavity surface-emitting laser (VCSEL), a light emitting diode (LED), an edge emitting diode laser, and a flash lamp.

The method further comprises delivering the intensity modulated sample light along the respective optical path in the anatomical structure during a single measurement period, such that the intensity modulated sample light is scattered by the anatomical structure, resulting in signal light that exits the anatomical structure, and detecting the respective signal light (e.g., using a photodiode) over the frequency range within the measurement period via each of the paired optical source-detector combinations.

In one method, the plurality of paired optical source-detector combinations is defined using a single optical source and multiple optical detectors, such that a different optical path is created between the single optical source and each respective optical detector. In another method, the plurality of paired optical source-detector combinations is defined using multiple optical sources and a single optical detector, such that a different optical path is created between each respective optical source and the single optical detector. In still another method, the plurality of paired optical source-detector combinations is defined using multiple optical sources and multiple optical detectors, such that different optical paths are created between each respective optical source and each respective optical detector.

In another method, detecting the intensity modulated signal light comprises detecting the signal light and outputting an electrical physiological-encoded signal, amplifying the physiological-encoded signal, and digitizing the amplified physiological-encoded signal into digital physiological-encoded data. Amplifying the physiological-encoded signal comprises outputting an intensity and phase of the physiological-encoded signal in response to an electrical signal output at the multiple frequencies, in which case, the intensity and phase is digitized into digital physiological-encoded data. The intensity modulated signal light may be detected with at least one discrete detector. Each of the discrete detector(s) may have an area greater than 30 μm², or even greater than 200 μm², but preferably less than 1000 μm².

The method further comprises analyzing the detected signal light for all of the paired optical source-detector combinations over the respective frequency ranges, and determining an occurrence and a location of a physiological event (e.g., a fast-optical signal) in at least two dimensions (which may include a spatial depth) in the anatomical structure based on the analysis.

In one method, the detected signal light for each paired optical source-detector combination is analyzed in the frequency domain at one or more frequencies, and the occurrence of the physiological event in the anatomical structure is based on the analysis in the frequency domain. For example, the occurrence of the physiological event in the anatomical structure may be determined by comparing a difference between the detected signal light for each paired optical source-detector combination to a baseline signal light (e.g., a user-specific model) at the one or more frequencies.

In another method, the detected signal light for each paired optical source-detector combination is analyzed in the time domain at one or more optical path lengths, and the occurrence of the physiological event in the anatomical structure is determined based on the analysis in the time domain. For example, the frequency domain representation of the detected signal light for each paired optical source-detector combination can be transformed into a time domain representation of the detected signal light (e.g., using an Inverse Fast Fourier Transform (IFFT)) to obtain intensity-optical path length information of the respective detected signal light, in which case, the occurrence of the physiological event in the anatomical structure may be determined based on intensity-optical path length information, e.g., by comparing a difference between the detected signal light (e.g., a user-specific model) for each paired optical source-detector combination to baseline signal light at the one or more optical path lengths.

In still another method, the detected signal light for each paired optical source-detector combination is analyzed in the time domain at one or more optical path lengths, and the spatial depth of the physiological event in the anatomical structure is determined based on the analysis in the time domain. For example, a frequency domain representation of the detected signal light for each paired optical source-detector combination can be transformed into a time domain representation of the detected signal light (e.g., using an Inverse Fast Fourier Transform (IFFT)) to obtain intensity-optical path length information of the detected signal light, wherein the spatial depth of the physiological event in the anatomical structure is determined based on intensity-optical path length information. The spatial depth of the physiological event in the anatomical structure may be determined by comparing a difference between the detected signal light for each paired optical source-detector combination to baseline signal light (e.g., a user-specific model) at the one or more optical path lengths.

In yet another method, the detected signal light for each paired optical source-detector combination is analyzed in the frequency domain at one or more frequencies, and the spatial depth of the physiological event in the anatomical structure is determined based on the analysis in the frequency domain. For example, the spatial depth of the physiological event in the anatomical structure is may be determined by comparing a difference between the detected signal light for each paired source-detector combination to baseline signal light (e.g., a user-specific model) at the one or more frequencies at the one or more frequencies.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of a single-source single-detector optical non-invasive measurement system constructed in accordance with one embodiment of the present inventions;

FIG. 2 is a frequency domain diagram of the intensity and phase of signal light detected by the optical non-invasive measurement system of FIG. 1;

FIG. 3 is a detailed block diagram of the single-source single-detector arrangement used in the optical non-invasive measurement system of FIG. 1;

FIG. 4 is a block diagram of a multi-source multi-detector optical non-invasive measurement system constructed in accordance with another embodiment of the present inventions;

FIG. 5 is a detailed block diagram of the multi-source multi-detector arrangement used in the optical non-invasive measurement system of FIG. 4;

FIG. 6 is a block diagram of a single-source multi-detector optical measurement system constructed in accordance with still another embodiment of the present inventions;

FIG. 7 is a detailed block diagram of the single-source multi-detector arrangement used in the optical non-invasive measurement system of FIG. 6;

FIG. 8 is a block diagram of a multi-source single-detector optical non-invasive measurement system constructed in accordance with yet another embodiment of the present inventions;

FIG. 9 is a detailed block diagram of the multi-source single-detector arrangement used in the optical non-invasive measurement system of FIG. 8;

FIG. 10A is a frequency domain diagram of the currently detected and baseline intensity and phase of signal light plotted for a paired optical source-detector combination of any of the optical non-invasive measurement systems of FIGS. 1, 4, 6, and 8;

FIG. 10B is a time domain diagram of the currently detected and baseline intensity and phase of signal light plotted for a paired optical source-detector combination of any of the optical non-invasive measurement systems of FIGS. 1, 4, 6, and 8;

FIG. 11 is a plan view of wearable and unwearable units in which the optical non-invasive measurement systems of FIGS. 1, 4, 6, and 8 may be embodied; and

FIG. 12 are profile views of one arrangement of the output port and input port of the wearable unit of FIG. 11, particularly illustrating the creation of an optical path in tissue between the ports;

FIG. 13 is a plan view illustrating an arrangement of a single movable output port and a single fixed input port for use in the optical non-invasive measurement system of FIG. 1, as embodied in the wearable unit of FIG. 11;

FIG. 14 is a plan view illustrating an arrangement of multiple fixed output ports and multiple fixed input ports for use in the optical non-invasive measurement system of FIG. 4, as embodied in the wearable unit of FIG. 11;

FIG. 15 is a plan view illustrating an arrangement of a single movable output port and multiple fixed input ports for use in the optical non-invasive measurement system of FIG. 6, as embodied in the wearable unit of FIG. 11;

FIG. 16 is a plan view illustrating an arrangement of a single movable input port and multiple fixed output ports for use in the optical non-invasive measurement system of FIG. 8, as embodied in the wearable unit of FIG. 11;

FIG. 17 is a flow diagram illustrating one method used by the optical measurement systems of FIGS. 1, 4, 6, and 8 to non-invasively detect and localize a fast-optical signal in brain tissue; and

FIG. 18 is a flow diagram illustrating one method used by the optical non-invasive measurement systems of FIGS. 1, 4, 6, and 8 to localize a fast-optical signal in brain tissue.

### DETAILED DESCRIPTION OF THE EMBODIMENTS

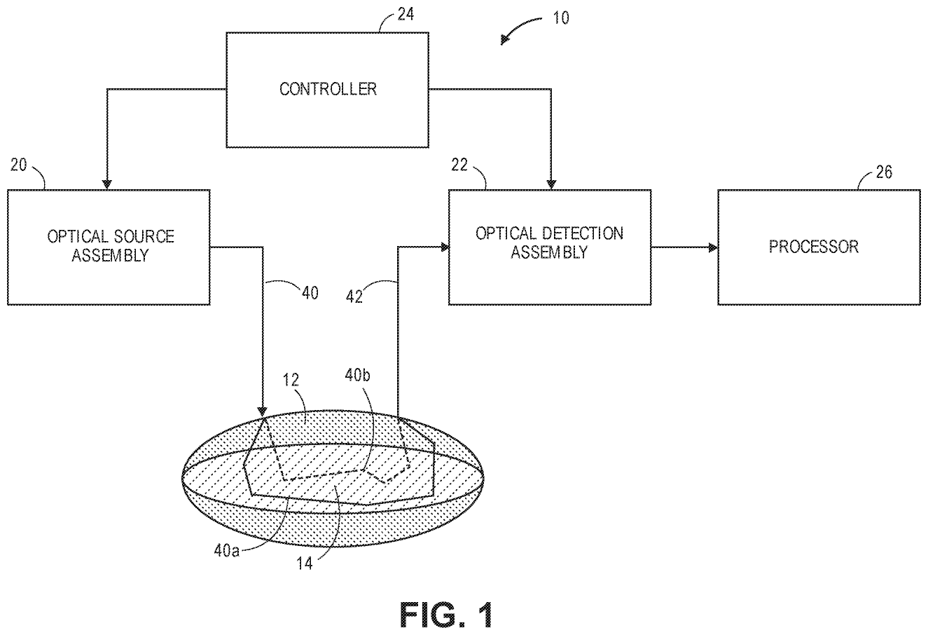

Referring first to FIG. 1, one embodiment of an optical non-invasive measurement system 10 constructed in accordance with the present inventions will now be described. The optical measurement system 10 is designed to non-invasively detect and localize a physiological event in an anatomical structure 12. In the illustrated embodiments, the anatomical structure 12 is a brain. Although for exemplary purposes, the optical measurement system 10 is described herein as being used to detect and localize a physiological event in brain tissue, variations of the optical measurement system 10 can be used to detect and localize a physiological event in other anatomical parts of a human body, animal body and/or biological tissue.

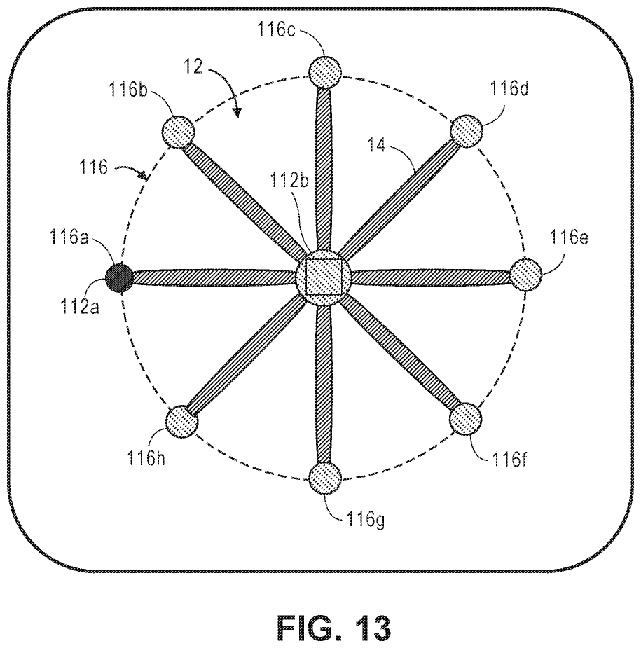

Although the optical non-invasive measurement system 10 is initially described as creating one optical path 14 through the brain 12, in a practical implementation, variations of the optical non-invasive measurement system 10 described herein will create multiple optical paths 14 spatially separated from each other within anatomical structure 12. Thus, it should be understood that the optical measurement systems described herein may be capable of creating more than one optical path 14 through the anatomical structure 12. For example, the simple source-detector arrangement of the optical measurement system 10, which can only create one optical path 14 within a measurement period, may be physically moved between the creation of optical paths 14 during multiple measurement periods, as shown in FIG. 13.

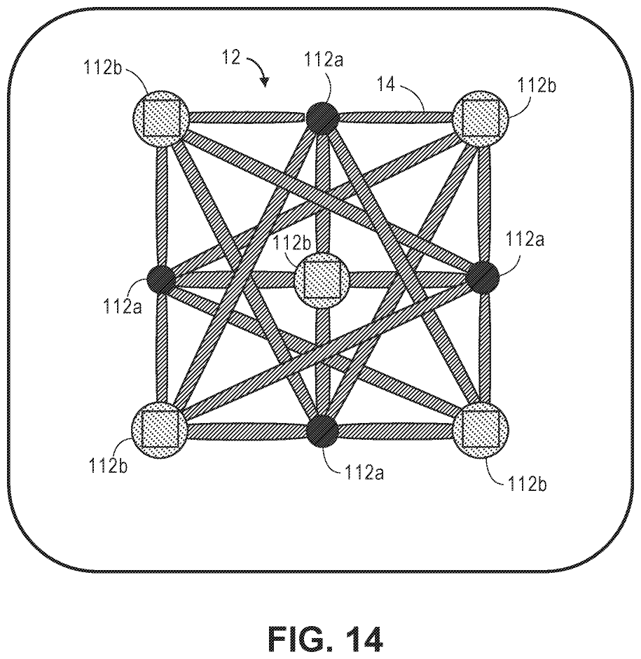

Further variations of the optical non-invasive measurement system 10 may utilize complex source-detector arrangements (e.g., single-source multi-detector, multi-source single-detector, or multi-source multi-detector) to simultaneously create multiple optical paths 14 during a single measurement period, and may also physically moved between the creation of optical paths 14 during multiple measurement periods to create additional optical paths 14, as shown in FIGS. 14-16. It is also possible to vary the frequency of one or more sources during a single measurement period of a single-source multi-detector, multi-source single-detector, or multi-source multi-detector arrangement. The choice between any of these types of arrangements will depend upon the particular use and form-factor of the optical non-invasive measurement system 10. For example, if the use only requires a line of information to be acquired, a fixed single source single-detector arrangement may be used. In contrast, if planes or volumes of information are to be acquired, a movable single-source single detector arrangement, a single-source multi-detector arrangement, a multi-source single-detector arrangement, or a multi-source multi-detector arrangement may be used.

In the illustrated embodiment, the optical non-invasive measurement system 10 detects neurological events that result in fast-optical signals (i.e., perturbations in the optical properties of neural tissue caused by mechanisms related to the depolarization of neural tissue, including, but not limited to, cell swelling, cell volume change, changes in membrane potential, changes in membrane geometry, ion redistribution, birefringence changes, etc.), although in alternative embodiments, the diffusive optical measurement non-invasive system 10 may alternatively or additionally be tuned to detect other physiological events that cause a change in an optical property of the brain 12, e.g., Doppler shift due to moving blood flow, changes in blood volume, metabolism variations such a blood oxygen changes. As will be described in further detail below, optical non-invasive measurement system 10, when properly tuned to a specific type of physiological event, and in this case, the presence of a fast-optical signal, is capable of decoding light propagating through the brain 12 to detect that physiological event.

Information and acquired neural data related to the detected physiological event may be used (e.g., computed, processed, stored, etc.) internally within the optical non-invasive measurement system 10 to adjust the detection parameters of the optical measurement system, such as increasing or decreasing the strength of the optical source and/or data compression and/or analysis, such a Fast Fourier Transform (FFT) and/or statistical analysis; or may be transmitted to external programmable devices for use therein, e.g., medical devices, entertainment devices, neuromodulation stimulation devices, lie detection devices, alarm systems, educational games, brain interface devices, etc.

Significantly, the optical non-invasive measurement system 10 provides a relatively simple means for detecting physiological events, such as fast-optical signals, in the brain with a relatively high sensitivity and at a sufficient spatial depth resolution. The technique used by the optical non-invasive measurement system 10 should be contrasted with the frequency domain diffuse optical tomography (DOT) approach and the frequency-domain photon migration (FDPM) approach discussed in the background of the invention, which do not detect fast-optical signals at a sufficient spatial depth resolution and sensitivity. The technique used by the optical non-invasive measurement system 10 should also be contrasted with the interferometric Near-Infrared Spectroscopy (iNIRS) approach discussed in the background of the invention, which can detect fast-optical signals at a sufficient spatial depth resolution, but does so using a relatively complicated and expensive arrangement of components (e.g., the requirement of a high-coherence optical source, reference beam, associated beam splitters and combiners, and a balanced detector).

The optical non-invasive measurement system 10 detects and localizes physiological events associated with neural activity in the brain, including fast-optical signals, in three-dimensions, with two of the dimensions represented as an x-y plane spanning the surface of the brain 12 being localized by creating multiple optical paths 14 (using a complex source-detector arrangement and/or by moving a simple source-detector arrangement) and the third dimension (z-dimension or depth into the brain 12) being localized by measuring the frequency response of the brain 12 to light intensity.

Significantly, the frequency response of the brain 12 is measured by intensity modulating sample light delivered into the brain 12 at many different frequencies (in comparison to existing approaches to fast-optical detection, such as Gratton, which use only one frequency) preferably extending into the gigahertz (GHz) range, e.g., up to 10 GHz. Doing so offers several benefits: (1) the detection sensitivity of physiological events, such as the fast-optical signal, is increased; and (2) the spatial information of the detected physiological event is improved (e.g., by conveniently deriving path-length-selective measurements of the detected physiological event from the frequency response information).

Specifically, using many closely spaced frequencies, rather than one or a few frequencies, allows a wide range of depths to be selectively probed, including large depths into brain tissue and beneath the skin and skull, while also providing for more sensitive detection of the fast-optical signals, in comparison with existing approaches to fast-optical detection, such as Gratton, which uses only one frequency. Moreover, extending the frequency range into very high frequencies, such as >10 GHz, allows for providing high spatial resolution along the depth direction, i.e., high specificity discrimination of path length. Together, these features can be viewed as allowing the frequency domain system to provide full characterization of the time of flight distribution of the photons after performing appropriate data analysis. This provides enhanced information on both fast-optical signal strength and on the depth or path length-resolved features of the past optical signal strength. Furthermore, because the frequency response technique used by the optical measurement system 10 does not require holography, in addition to not requiring complex and expensive equipment, the optical non-invasive measurement system 10 does not require the detection of speckles (i.e., the use of highly coherent light and the ability to spatially resolve speckles at the detection plane). As such, it is possible for the current system to utilize very simple optical sources that are partially coherent (e.g., LEDs or VCSEL diodes), as well as large and simple photodiodes to detect this partially coherent light across a large area, thus collecting many more photons per detector than in the case of spatially resolved speckle.

Returning to FIG. 1, the optical non-invasive measurement system 10 generally comprises an optical source assembly 20, an optical detection assembly 22, a controller 24, and a processor 26, which operate together to non-invasively detect and localize a fast-optical signal in the brain 12.

The optical source assembly 20 is configured for intensity modulating sample light 40 at multiple frequencies within a frequency range, and delivering the intensity modulated sample light 40 along the optical path 14 in the brain 12 during a single measurement period, such that the intensity modulated sample light 40 scatters diffusively, e.g., through the human skull, into the brain, and back out again, exiting as signal light 42. As it scatters diffusively through the brain 12, various portions of the sample light 40 will take different paths through the brain 12. For purposes of brevity, only a first sample light portion 40a traveling along a relatively long path, and a second sample light portion 40b traveling along a relatively short path, are illustrated, although it should be appreciated that the diffused sample light 40 will travel along many more paths through the brain 12.

Significantly, the sample light portions 40a, 40b travel along the optical path 14 and exit the brain 12 as the signal light 42, which is encoded with any physiological events that change an optical property along the optical path 14 of the brain 12. As will be described in further detail below, the optical non-invasive measurement system 10 is capable of spatially distinguishing the sample light portions 40a, 40b from each other, and thus determining the depth of a physiological event, based on the frequency response of the tissue in the brain 12. It should be appreciated that, although not all of the sample light 40 from which the signal light 42 is derived passes through the brain 12 and is detected, it is only important that at least some of the signal light 42 exiting the brain 12 be detected.

The sample light 40, and thus the signal light 42, may be ultraviolet (UV) light, visible light, and/or near-infrared and infrared light, and may have any suitable wavelength, e.g., in the range of 350 nm-1800 nm. The sample light 40 may be close to monochromatic in nature, comprising approximately a single-wavelength light, or the sample light 40 may have multiple wavelengths (e.g., white light). In some variations, the sample light 40 may have a broad optical spectrum or may have a narrow optical spectrum that is then rapidly swept (e.g., changed over time) to functionally mimic or create an effective broad optical spectrum.

Notwithstanding the foregoing, it is preferred that the optical wavelength of the sample light 40 be selected to maximize sensitivity to the specific physiological event of interest. For example, in the preferred case where the physiological event of interest is the presence of a fast-optical signal, an optical wavelength greater than 850 nm may be used for the sample light 40. Optionally, an optical wavelength equal to or greater 1000 nm may be used for the sample light 40 to maximize penetration. In the additional or alternative case where the physiological event of interest is a change in the blood oxygen concentration, an optical wavelength in the range of 650 nm to 750 nm may be used for the sample light 40. Multiple optical wavelengths can be used for the sample light 40 to allow different physiological events to be distinguished from each other. For example, sample light 40 having two optical wavelengths of 900 nm and 700 nm can be respectively used to resolve fast-optical signals and blood oxygenation. Alternatively, the wavelength of the sample light 40 to be selected to maximize the detector sensitivity.

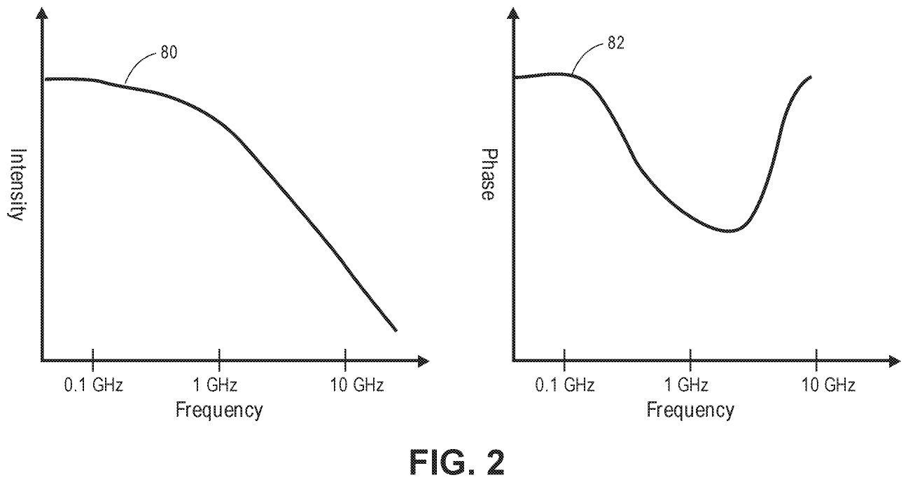

As will be described in further detail below with respect to FIG. 2, the optical source assembly 20 comprises control inputs for receiving control signals from the controller 24 that instruct the optical source assembly 20 to emit the sample light 40 at a selected time, duration, and intensity, as well as at one or more intensity modulation frequencies. In the preferred embodiment, the controller 24 instructs the optical source assembly 20 to serially intensity modulate the sample light 40 respectively at multiple frequencies, e.g., by instructing the optical source assembly 20 to sweep the frequency at which the sample light 40 is intensity modulated (e.g., by “chirping”), although the frequency at which the sample light 40 is serially intensity modulated may be otherwise discretely varied (e.g., randomly or otherwise modified in accordance with a defined frequency switching pattern that jumps between frequencies). The time duration that the sample light 40 is emitted for each frequency may depend on the signal-to-noise ratio (SNR) of the resulting signal light 42 at that frequency. That is, the less the SNR of the resulting signal light 42 at any particular frequency, the greater the emission time of the sample light 40 for that frequency. In a further alternative embodiment, the controller 24 may instruct the optical source assembly 20 to simultaneously intensity modulate the sample light 40 at multiple frequencies (i.e., sample light 40 can be modulated with multiple frequencies in parallel).

The optical detection assembly 22 is configured for, over the frequency range, detecting the signal light 42 and outputting a complex frequency spectrum measurement (i.e., intensity and phase) of the detected signal light 42 within the measurement period. For example, exemplary intensity profile information 80 and phase profile information 82 (the phase being measured by assigning a phase to the detected intensity of the signal light 42 versus time curve with respect to the phase of the sample light 40 for each frequency) of the detected signal light 42 over a frequency spectrum ranging from 0.1 GHz to 10 GHz may be output by the optical detection assembly 22, as respectively illustrated in FIG. 2.

In this embodiment, where there is a simple source-detector arrangement, only one set of frequency spectrum information (intensity profile information 80 and phase profile information 82) will be detected for each measurement period, although as will be described in further detail below, when using a complex source-detector arrangement, multiple sets of frequency spectrum information will be detected for each measurement period. As will be described in further detail below, the processor 26 can use the intensity profile information 80 and phase profile information 82 of the detected signal light 42 to both determine the occurrence and spatial depth (z-dimension) of a fast-optical signal in the brain 12, and can further use the geometric information of spatially resolved paired source-detector combinations to determine the location of the fast-optical signal along the x-y plane (i.e., plane relative to the surface of the brain 12).

It should be appreciated that, because the optical measurement system 10 does not utilize holography, the measurement period may have a duration longer than the “speckle decorrelation time” of the tissue in the brain 12. The speckle decorrelation time is due to the scatters' motion (for example, blood flow) inside living biological tissue, and rapidly decreases with the depth at which the tissue is to be imaged, and in particular, scales super-linearly with the depth into the brain 12 at which the optical path 14 is located, falling to microseconds or below as the measurement depth extends to the multi-centimeter range. Thus, the duration of the measurement period need only be as short as the physiological event intended to be detected (in this case, a fast-optical signal), thereby decreasing the hardware constraints placed on the optical detection assembly 22.

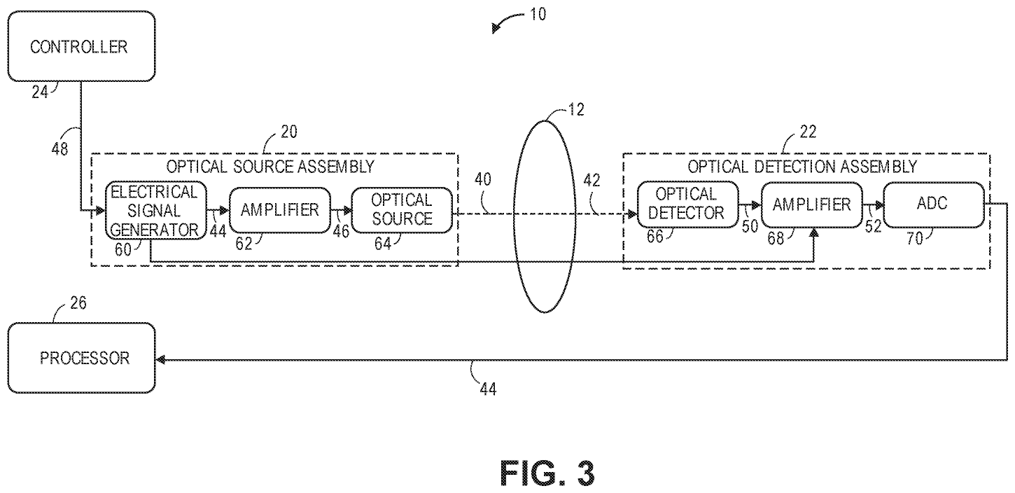

As will be discussed in further detail below, the optical detection assembly 22 can be locked to an intensity modulation frequency of the signal light 42 at any given time to maximize the SNR of the signal light 42, and to this end, may comprise control inputs for receiving control signals directly or indirectly from the controller 24 that allow the optical detection assembly 22 to detect the signal light 42 at the specific intensity modulation frequency, as will be described in further detail below in FIG. 3. Thus, as the intensity modulation frequency of the sample light 40 is serially varied by the optical source assembly 20, the optical detection assembly 22 will be serially locked to the different intensity modulation frequencies. If, alternatively, the sample light 40 is simultaneously intensity modulated at the multiple frequencies, the optical detection assembly 22 may be simultaneously locked to the different intensity modulation frequencies, and a superposition of the intensities and phases of the signal light 42 can be output, which can subsequently be de-mixed during processing of the signal light 42.

Referring further to FIG. 3, one detailed embodiment of the optical non-invasive measurement system 10 will now be described. The optical source assembly 20 comprises an electrical signal generator 60 configured for outputting an electrical alternating current (AC) signal 44 at the multiple frequencies (corresponding to the intensity modulation frequencies of the sample light 40); an amplifier 62 configured for amplifying the AC signal 44 and outputting a drive signal 46; and an optical source 64 configured for outputting the intensity modulated sample light 40 at the multiple frequencies in accordance with the drive signal 46. The intensity modulated sample light 40 may then be delivered into the anatomical structure (in this case, the brain 12), which is scattered as signal light 42 that exists the brain 12, as described above.

The electrical signal generator 60 may receive control signals 48 from the controller 24 (either analog or direct digital synthesis inputs) for setting the frequencies of the AC signal 44 at which the sample light 40 is intensity modulated. If the sample light 40 is serially intensity modulated at the respective multiple frequencies, the frequency of the AC signal 44 output by the electrical signal generator 60 will likewise be serially varied. If the sample light 40 is simultaneously intensity modulated at the respective multiple frequencies, the AC signal 44 output by the electrical signal generator 60 will simultaneously have the multiple frequencies. Alternatively, a direct current (DC) offset (not shown) can be applied to bias the optical source 64 to allow it to more quickly turn on and off. It should be appreciated that the drive signal 46 may not be sinusoidal due to the diode nature of the optical source 64 (in some cases), but may be triangular or on-linear to achieve the desired sinusoidal waveform for the sample light 40 in a preferred implementation.

Advantageously, because the optical non-invasive measurement system 10 does not utilize holography, the optical source 64 may take the form of a very simple and inexpensive component, such as a vertical-cavity surface-emitting laser (VCSEL), a light emitting diode (LED), an edge emitting diode laser, a flash lamp, etc. Preferably, the optical source 64 is a high-coherence light source (i.e., a laser), although in alternative embodiments, the optical source 64 may be a low-coherence light source.

In the illustrated embodiment, the optical source 64 is a pulsed wave (PW) optical source that is alternately turned on and off by the drive signal 46. In this case, the on/off frequency of the AC signal 44 may be serially varied (e.g., sweeping or discretely varying the frequency) by appropriate control signals by the controller 24, thereby serially varying the frequency of the intensity modulated sample light 40 output by the optical source 64. Alternatively, the optical source 64 may be a continuous wave (CW) optical source, in which case, the sample light 40 output by the optical source 64 may be passed through an intensity modulator (not shown), such as an electro-optic modulator or quantum well modulator, or the sample light 40 may be bent in a time-varying manner, e.g., via an acousto-optic or micro-electrical-mechanical system (MEMS). In any event, the instantaneous oscillation of the intensity modulated sample light 40 output by the optical source 64 may be set by the controller 24 by sending appropriate control signals to the optical source assembly 20.

The optical detection assembly 22 comprises an optical detector 66 configured for detecting the exiting signal light 42 and outputting an electrical physiological-encoded signal 50 representative of the intensity modulated signal light 42 that is encoded with any physiological events that may perturb the sample light 40; an amplifier 68 configured for amplifying the physiological-encoded signal 50, and an analog-to-digital converter (ADC) 70 configured for digitizing the amplified signal 52 into digital physiological-encoded data 54, which is sent to the processor 26 for processing, as will be described in further detail below.

Advantageously, because the optical non-invasive measurement system 10 does not utilize holography, and therefore, need not detect speckle grains, the optical detector 66 may take the form of a very simple and inexpensive single discrete component (e.g., a photodiode). The optical detector 66 may be relatively large compared to camera pixels in holography systems in order to maximize collection of photons from the signal light 42, e.g., having an area greater than 30 μm², or even an area greater than 200 μm². Of course, the size of the optical detector 66 should be limited, e.g., less than 1000 μm², such that the form factor of the optical measurement system 10 may be minimized, especially in the alternative embodiment where multiple optical detection assemblies 22 are utilized. Alternatively, the optical detector 66 may comprise several discrete components to suppress shot noise and achieve fast photodetector bandwidths that operate in the GHz regime. Ultimately, the size of the optical detector 66 and number of discrete components that make up the optical detector 66 may be determined by the required number of photons captured during the measurement period due to the need to suppress shot noise and by the need to achieve fast photodetector bandwidths that operate in the GHz regime, e.g., sufficiently low capacitance (i.e., as the size of the optical detector 66 increases, it will have more capacitance, and will thereby have a slower response that will reduce its ability to measure the response, e.g., greater than 10 GHz).

In the illustrated embodiment, the amplifier 68 advantageously takes the form of a lock-in amplifier, which in general, is any device that can extract the intensity and phase of a sinusoidally varying component, while removing a potentially large direct current (DC) background, as well as components of a signal at frequencies other than the frequency to which it is locked. Thus, the amplifier 68, as a lock-in amplifier, will be locked to the frequency of the AC signal 44 at any given point in time, and thus, the frequency at which the sample light 40 is intensity modulated. That is, the amplifier 68 will be configured for, in response to the AC signal 44 output by the electric signal generator 60 at the defined frequency, amplifying the physiological-encoded signal 50 at the defined frequency, and outputting an intensity and phase of the amplified signal 52, which is then digitized by the ADC 70. Significantly, due to the use of a lock-in amplifier 68, as compared to a broadband amplifier, the amplified signal 52 will have much less noise, which greatly facilitates the ability to intensity modulate the sample light 40 at higher frequencies.

In the context of the optical non-invasive measurement system 10, which advantageously utilizes high intensity modulation frequencies in the GHz range to increase detection sensitivity of the signal light 42, the use of a lock-in amplifier can be used to enable the relatively small intensity signal light 42 at these high intensity modulation frequencies, which have attenuated by the fall-off of the tissue response at these high modulation frequencies, to nevertheless be extracted. In addition, the controller 24 may adaptively set the amount of integration time used by the lock-in amplifier at each frequency in order to obtain an acceptable SNR for both intensity and phase, even at strongly attenuated high modulation frequencies. A lock-in amplifier can be implemented, e.g., with fast shuttering or optical modulation mechanism, or with an electronic multiplier circuit coupled with fixed or variable electronic frequency generators, pre-amplifiers, and electronic low-pass filters, e.g., implemented through resistor-capacitor-inductor circuits. For example, the lock-in amplifier may be fabricated as parts of integrated application specific integrated circuits (ASICs), and may be integrated in a monolithic silicon integrated circuit.

Although the use of a lock-in amplifier 68 maximizes the SNR of the signal light 42, in alternative embodiments, the amplifier 68 may not be a lock-in amplifier, but rather broadly amplifies the detected signal light 42. However, the SNR of the signal light 42 will generally decrease in this case.

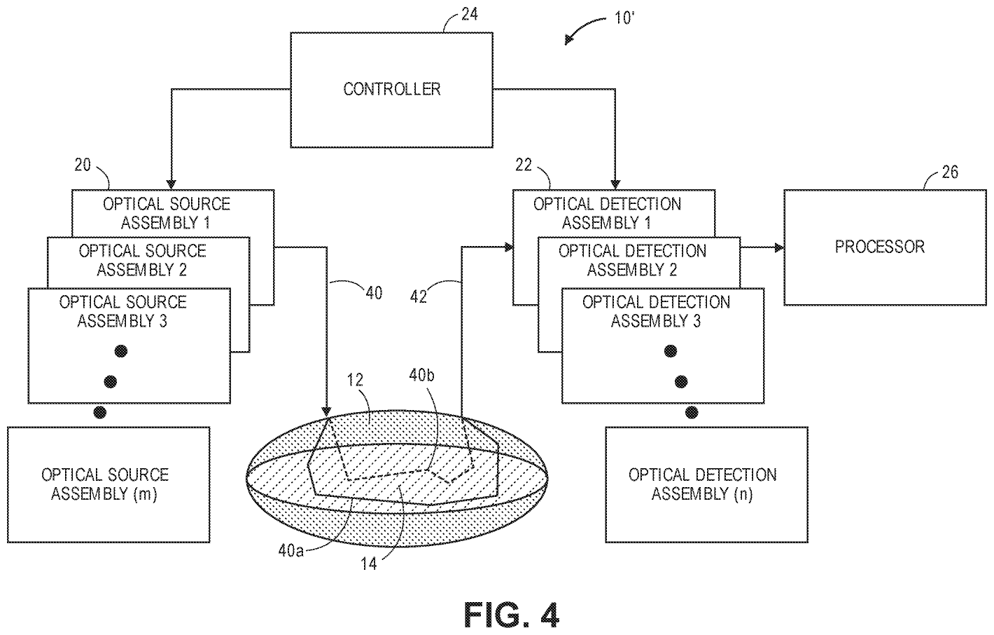

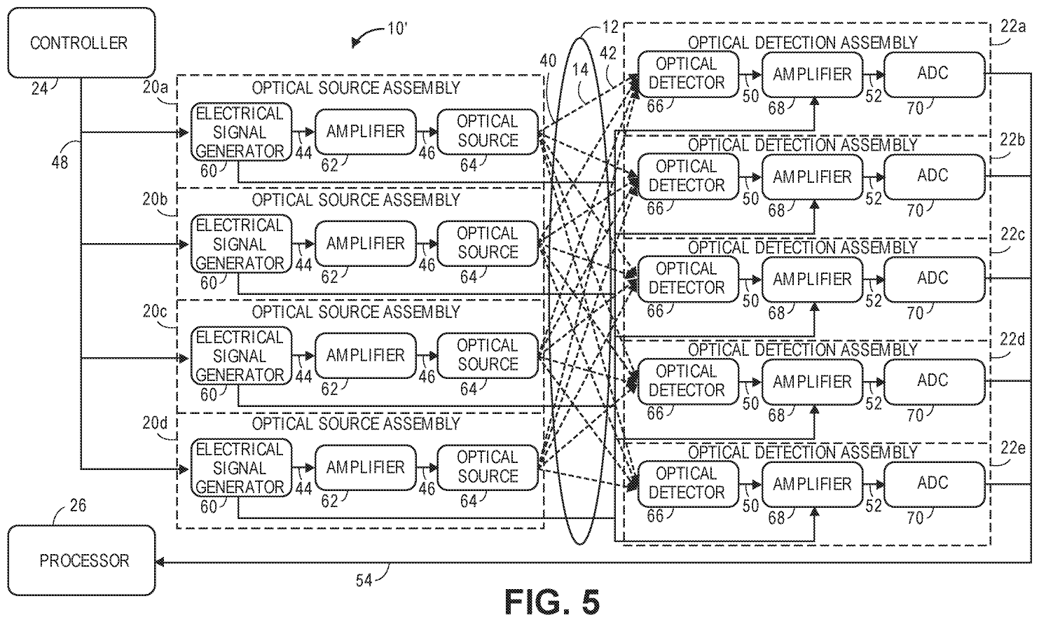

In an optional embodiment illustrated in FIG. 4, an optical non-invasive measurement system 10′ has a multiple source-detector arrangement (in this case, multi-source multi-detector), such that different optical paths 14 (geometric paths) are defined between each of an m number of respective optical source assemblies 22 and an n number of respective optical detection assemblies 24 to create an m×n number of different paired optical source-detector combinations, thereby facilitating localization of the fast-optical signal along the surface of the brain. For example, if the optical non-invasive measurement system 10′ comprises four optical source assemblies 20a-20d and five optical detection assemblies 22a-22e, as illustrated in FIG. 5, twenty paired source-detector combinations (or twenty different geometric paths) may be simultaneously created, thereby allowing twenty corresponding sets of frequency spectrum information (see FIG. 2) to be generated in a single measurement period.

In a similar manner described above with respect to the single source-detector arrangement of the optical non-invasive measurement system 10, each optical source assembly 20, under control of the controller 24, is configured for intensity modulating sample light 40 at multiple frequencies within a frequency range, and delivering the intensity modulated sample light 40 along a respective optical path 14 in the brain 12 during a single measurement period, such that the intensity modulated sample light 40 scatters diffusively, e.g., through the human skull, into the brain, and back out again, exiting as signal light 42; and each optical detection assembly 22, under control of the controller 24, is configured for, over the frequency range, detecting the signal light 42 and outputting an intensity and phase of the detected signal light 42 within the measurement period.

Thus, assuming four optical source assemblies 20a-20d and five optical detection assemblies 22a-22e, as illustrated in FIG. 5, during a single measurement period and over the entire frequency range, the optical detection assembly 22a will detect the signal light 42 resulting from the sample light 40 delivered by each of the optical source assemblies 20a-20d; the optical detection assembly 22b will detect the signal light 42 resulting from the sample light 40 delivered by each of the optical source assemblies 20a-20d; the optical detection assembly 22c will detect the signal light 42 resulting from the sample light 40 delivered by each of the optical source assemblies 20a-20d; the optical detection assembly 22d will detect the signal light 42 resulting from the sample light 40 delivered by each of the optical source assemblies 20a-20d; and the optical detection assembly 22e will detect the signal light 42 resulting from the sample light 40 delivered by each of the optical source assemblies 20a-20d.

It is preferred that the intensity modulation frequencies of the sample light 40 delivered by all of the optical source assemblies 20a-20d differ from each other at any given time, so that the resulting signal light 42 detected by each respective optical detection assembly 22 can be frequency distinguished, and thus, be associated with the correct optical paths 14 (geometric paths) between the optical source assemblies 20a-20d and optical detection assemblies 22a-22e. If the time spent at certain frequencies that have a lower SNR are greater than at other frequencies with higher SNR, it is preferred that the intensity modulation frequency of the sample light 40 for each optical source assembly 20 be serially varied over the respective frequency range for the respective optical source assembly 20. That is, sample light 40 may be emitted by the optical source assemblies 20a-20d in parallel, but the sample light 40 emitted by each optical source assembly 20 is intensity modulated in a serial fashion to complete the entire frequency range for that optical source assembly 20.

For example, if the frequency range of interest is between 500 MHz and 10 GHz with twenty equality spaced steps (i.e., 0.5 GHz, 1 GHz, 1.5 GHz, etc.), the intensity modulation frequencies of the sample light 40 for the four optical source assemblies 20a-20d may be sufficiently spaced apart as 500 MHz, 2 GHz, 6 GHz, and 9 GHz at a particular time, so that resulting signal light 42 detected by the optical detection assemblies 22 can be properly associated with the geometrical paths. That is, signal light 42 detected at 500 MHz can be associated with the five geometric paths between the optical source assembly 20a and the five respective optical detection assemblies 22a-22e; signal light 42 detected at 2 GHz can be associated with the five geometric paths between the optical source assembly 20b and the five respective optical detection assemblies 22a-22e; signal light 42 detected at 6 GHz can be associated with the five geometric paths between the optical source assembly 20c and the five respective optical detection assemblies 22a-22e; and signal light 42 detected at 9 GHz can be associated with the five geometric paths between the optical source assembly 20d and the five respective optical detection assemblies 22a-22e).

Of course, as a natural consequence of varying the intensity modulation frequencies of the optical source assemblies 20a-20d of the frequency range, the intensity modulation frequencies will differ from 500 MHz, 2 GHz, 6 GHz, and 9 GHz at different times. However, it is only important that the intensity modulation frequencies for the respective optical source assemblies 20a-20d be varied, such that the four intensity modulation frequencies are not the same for any point in time to allow proper association of the detected signal light 42 with the geometric paths.

Referring further to FIG. 5, one detailed embodiment of the optical non-invasive measurement system 10′ will now be described. In this case, the circuitry of the single optical source assembly 20 and single optical detection assembly 22 illustrated in FIG. 3 can be respectively duplicated for the corresponding optical source assemblies 20a-20d and optical detection assemblies 22a-22e illustrated in FIG. 5.

That is, each of the optical source assemblies 20a-20d comprises an electrical signal generator 60 configured for outputting an electrical AC signal 44 at the multiple frequencies (corresponding to the intensity modulation frequencies of the sample light 40); an amplifier 62 configured for amplifying the AC signal 44 and outputting an AC signal 46; and an optical source 64 configured for outputting the intensity modulated sample light 40 at the multiple frequencies in accordance with the AC drive signal 46, which is then delivered into the brain 12.

Each of the optical detection assemblies 22a-22e comprises an optical detector 66 configured for detecting the exiting signal light 42 and outputting an electrical physiological-encoded signal 50 representative of the intensity modulated signal light 42 that is encoded with any physiological events that may perturb the sample light 40; an amplifier 68 configured for amplifying the physiological-encoded signal 50, and an ADC 70 configured for digitizing the amplified signal 52 into digital physiological-encoded data 54, which is sent to the processor 26 for processing.

If lock-in amplifiers are used, the amplifier 58 for each optical detection assembly 22 will comprise multiple lock-in amplifiers (one for each optical source assembly 20, and in this case four lock-in amplifiers), so that each optical detection assembly 22 can simultaneously lock into the frequencies at which the respective optical source assemblies 20a-20d intensity modulate the source light 40. Thus, each lock-in amplifier 68 within a respective one of the optical detection assembly 22 will be configured for, in response to the AC signal 44 output by the electric signal generator 60 of the corresponding optical source assembly 20 at the defined frequency, amplifying the physiological-encoded signal 50 at the defined frequency and outputting an intensity and phase of the physiological-encoded signal 50, which is then digitized by the ADC 70 into the digital physiological-encoded signal data 54.

Thus, as will be described in further detail below, two-dimensional spatial information (x- and y-spatial information along the surface of the brain) can be geometrically derived from multiple optical paths 14 (geometric paths) without requiring the optical source assemblies 20a-20d and optical detection assemblies 22a-22e to be physically moved relative to each other.

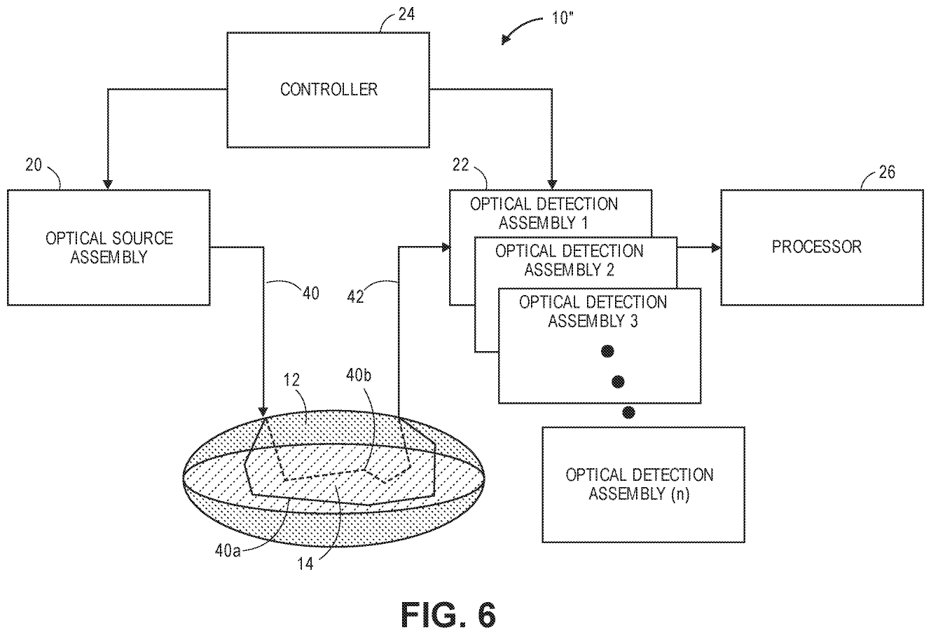

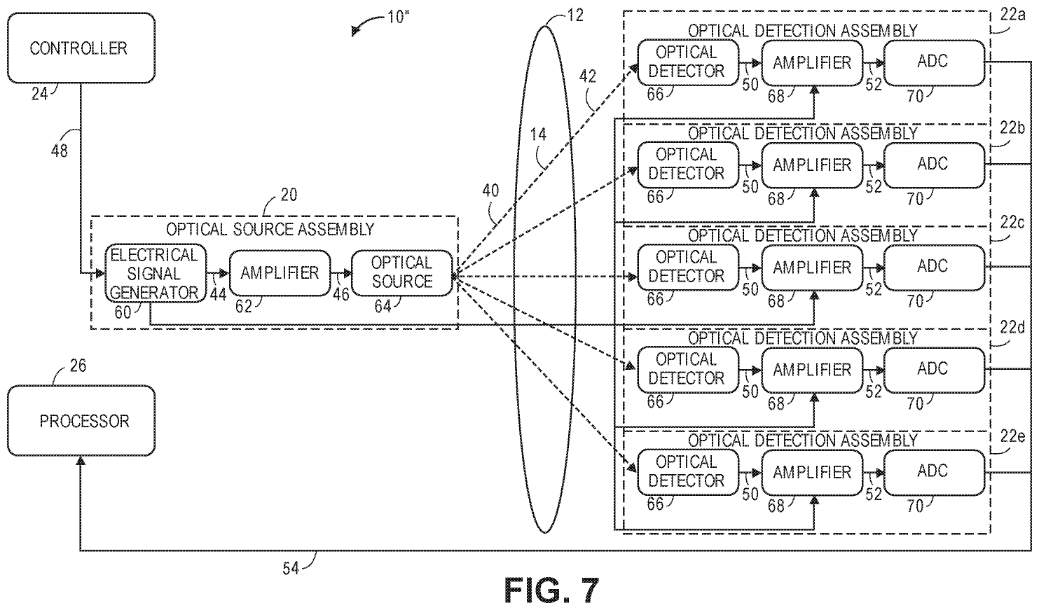

In another optional embodiment illustrated in FIG. 6, an optical non-invasive measurement system 10″ has a multiple source-detector arrangement (in this case, single-source multi-detector), such that different optical paths 14 (geometric paths) are defined between the single optical source assembly 22 and each of an n number of respective optical detection assemblies 24 to create a 1×n number of different paired optical source-detector assembly combinations, thereby facilitating localization of the fast-optical signal along the surface of the brain. For example, if the optical non-invasive measurement system 10″ comprises five optical detection assemblies 22a-22e, as illustrated in FIG. 7, five paired source-detector combinations (or five geometric paths) may be simultaneously created, thereby allowing five corresponding sets of frequency spectrum information (see FIGS. 2a and 2b) to be generated.

In a similar manner described above with respect to the single source-detector arrangement of the optical non-invasive measurement system 10, the optical source assembly 20, under control of the controller 24, is configured for intensity modulating sample light 40 at multiple frequencies within a frequency range, and delivering the intensity modulated sample light 40 along a respective optical path 14 in the brain 12 during a single measurement period, such that the intensity modulated sample light 40 scatters diffusively, e.g., through the human skull, into the brain, and back out again, exiting as signal light 42; and each of the optical detection assemblies 22a-22e, under control of the controller 24, is configured for, over the frequency range, detecting the signal light 42 and outputting an intensity and phase of the detected signal light 42 within the measurement period.

Referring further to FIG. 7, one detailed embodiment of the optical non-invasive measurement system 10″ will now be described. In this case, the circuitry of the single optical source assembly 20 in FIG. 3 can be identical to the circuitry of the single optical source assembly 22 illustrated in FIG. 7, while the circuitry of the single optical detection assembly 22 illustrated in FIG. 3 can be duplicated for the corresponding optical detection assemblies 22a-22e illustrated in FIG. 7.

That is, each of the optical detection assemblies 22a-22e comprises an optical detector 66 configured for detecting the exiting signal light 42 and outputting an electrical physiological-encoded signal 50 representative of the intensity modulated signal light 42 that contains a measure of physiological events that may perturb the sample light 40; an amplifier 68 configured for amplifying the physiological-encoded signal 50, and an ADC 70 configured for digitizing the amplified signal 52 into digital physiological-encoded data 54, which is sent to the processor 26 for processing.

Thus, two-dimensional spatial information (x- and y-spatial information along the surface of the brain) can be geometrically derived from multiple optical paths 14 (geometric paths) without requiring the optical source assembly 20 and optical detection assemblies 22a-22e to be physically moved relative to each other, as will be described in further detail below, although it may be desirable to physically move the optical source assembly 20 relative to the optical detection assemblies 22a-22e to increase the number of optical paths 14 (geometric paths), and thus, provide additional two-dimensional spatial information.

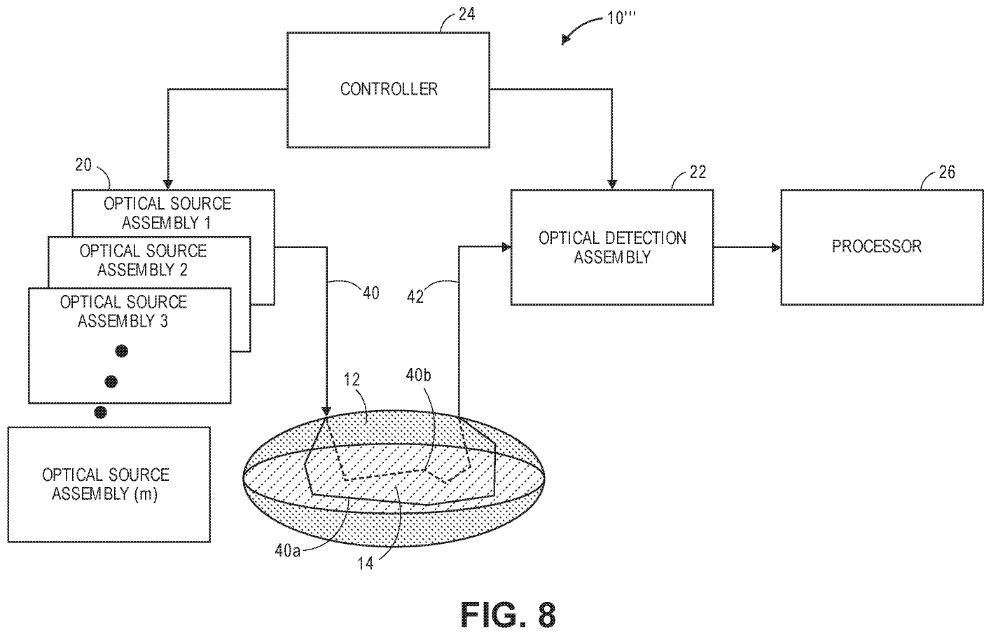

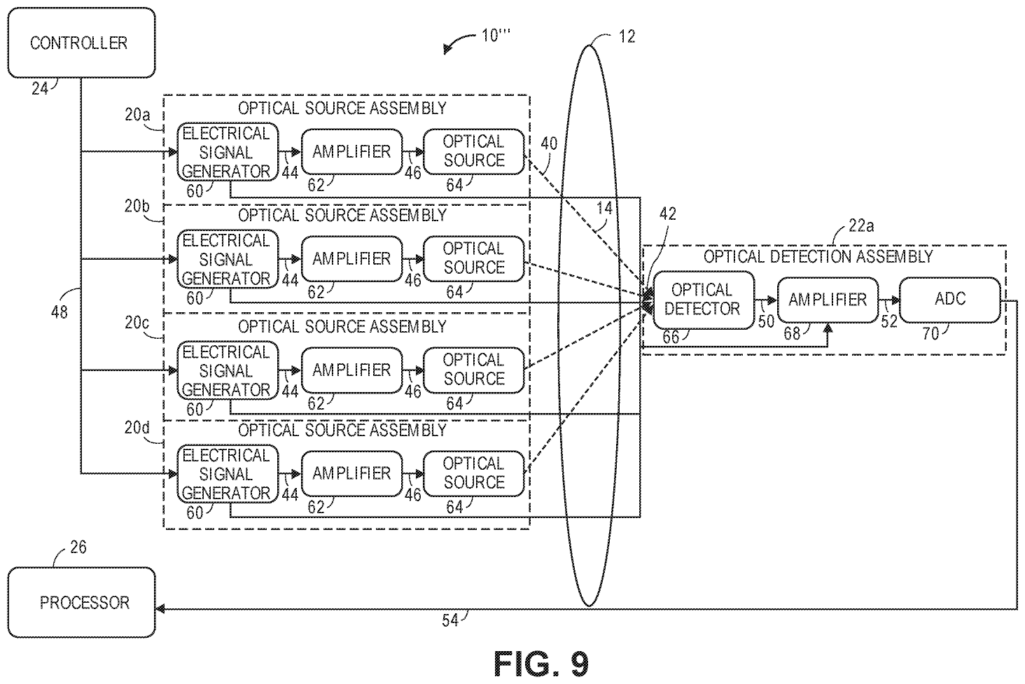

In another optional embodiment illustrated in FIG. 8, an optical non-invasive measurement system 10′″ has a multiple source-detector arrangement (in this case, multi-source single-detector), such that different optical paths 14 (geometric paths) are defined between an m number of respective optical source assemblies 22a-22d and the single optical detection assembly 24 to create an m×1 number of different paired optical source-detector assembly combinations, thereby facilitating localization of the fast-optical signal along the surface of the brain. For example, if the optical non-invasive measurement system 10″ comprises five optical source assemblies 22a-22d, as illustrated in FIG. 9, five paired source-detector combinations (or five geometric paths) may be simultaneously created, thereby allowing five corresponding sets of frequency spectrum information (see FIG. 2) to be generated.

In a similar manner described above with respect to the single source-detector arrangement of the optical non-invasive measurement system 10, each of the optical source assemblies 20a-20d, under control of the controller 24, is configured for intensity modulating sample light 40 at multiple frequencies within a frequency range, and delivering the intensity modulated sample light 40 along a respective optical path 14 in the brain 12 during a single measurement period, such that the intensity modulated sample light 40 scatters diffusively, e.g., through the human skull, into the brain, and back out again, exiting as signal light 42; and the single detection assembly 22, under control of the controller 24, is configured for, over the frequency range, detecting the signal light 42 and outputting an intensity and phase of the detected signal light 42 within the measurement period.

Thus, during a single measurement period and over the entire frequency range, the optical detection assembly 22 will detect the signal light 42 resulting from the sample light 40 delivered by the five optical source assemblies 20a-22e. Again, it is preferred that the intensity modulation frequencies of the sample light 40 delivered by all of the optical source assemblies 20a-20d differ from each other at any given time, so that the resulting signal light 42 detected by each respective optical detection assembly 22 can be frequency distinguished, and thus, be associated with the correct geometric paths between the optical source assemblies 20a-20d and optical detection assembly 22.

Referring further to FIG. 9, one detailed embodiment of the optical non-invasive measurement system 10′″ will now be described. In this case, the circuitry of the single optical source assembly 20 illustrated in FIG. 3 can be duplicated for the corresponding optical source assemblies 20a-20d illustrated in FIG. 9, while the circuitry of the single optical detection assembly 22 illustrated in FIG. 3 can be identical to the single optical detection assembly 22 illustrated in FIG. 9.

That is, each of the optical source assemblies 20a-20d comprises an electrical signal generator 60 configured for outputting an electrical AC signal 44 at the multiple frequencies (corresponding to the intensity modulation frequencies of the sample light 40); an amplifier 62 configured for amplifying the AC signal 44 and outputting an AC signal 46; and an optical source 64 configured for outputting the intensity modulated sample light 40 at the multiple frequencies in accordance with the AC drive signal 46, which is then delivered into the brain 12.

Thus, two-dimensional spatial information (x- and y-spatial information along the surface of the brain) can be geometrically derived from multiple optical paths 14 (geometric paths) without requiring the optical source assemblies 20a-20d and optical detection assembly 22 to be physically moved relative to each other, as will be described in further detail below, although it may be desirable to physically move the optical detection assembly 22 relative to the optical source assemblies 20a-20d to increase the number of optical paths 14 (geometric paths), and thus, provide additional two-dimensional spatial information.

Referring back to FIG. 1, the processor 26 is configured for analyzing the detected signal light 42 during the measurement period for all paired optical source-detector combinations (generated from the single paired source-detector arrangement of the optical non-invasive measurement system 10 illustrated in FIGS. 1 and 3 or the multiple paired source-detector arrangements of the optical non-invasive measurement systems 10′, 10″, and 10″ illustrated in FIGS. 4-9) over the frequency range(s). Based on this analysis, the processor 26 is further configured for determining an occurrence and a location of a fast-optical signal (as the physiological event) in at least two dimensions, and preferably three dimensions (including the spatial depth), within the brain 12 (as the anatomical structure). The processor 26 may perform post-processing on the detected fast-optical signal to generate additional information on the brain 12 along the optical path 14. For example, the processor 26 may determine a level of neural activity within the brain 12 along the optical path 14 based on the detected fast-optical signal (i.e., there will be neural activity at the location of the fast-optical signal).

Significantly, the processor 26 utilizes the frequency spectrum of the detected signal light 42 to determine both the occurrence and spatial depth (z-dimension) of the fast-optical signal in the brain 12 along the optical path 14, while utilizing the combination of the intensity of the signal light 42 and geometric information of the locations of the paired source-detector arrangements to obtain the x- and y-dimensions (along the surface of the brain) of the fast-optical signal within the brain 12.

Notably, the spatial resolution of the localization in z-dimension depends on the frequency parameters (i.e., the frequency range and frequency step size). In particular, the higher the frequency range extends, the finer the intensity and phase profile information will be in the frequency domain, and thus, the more spatial information in the z-dimension can be acquired. Furthermore, the frequency range over which the sample light 40 is intensity modulated must be fine enough to avoid “aliasing.” Thus, the frequency sampling must be selected to provide adequate resolution for the spatial information, while avoiding aliasing. It is preferred that the frequency range in which the sample light 40 is intensity modulated extend well into the GHz range in order to provide the sufficient resolution in the frequency domain. For example, the frequency range may comprise a frequency equal to or greater than 2 GHz, and preferably, a frequency equal to or greater than 5 GHz, and may extend from 1 GHz to 5 GHz, or even from 100 MHz to 10 GHz.

The spatial resolution of the localization in the x- and y-dimensions depends on the resolution of the geometric paths of the optical source-detector assembly combinations that can be created in the optical non-invasive measurement system 10. That is, the more paired optical source-detector assembly combinations that can be created in the optical non-invasive measurement system 10, the greater the spatial resolution of the localization in the x- and y-dimensions.

The processor 26 may utilize the frequency spectrum of the detected signal light 42 to determine the occurrence and spatial depth of the fast-optical signal within the brain 12 along the optical path 14, by analyzing the signal light 42 in the frequency domain at one or more frequencies and/or time domain at one or more optical path lengths.

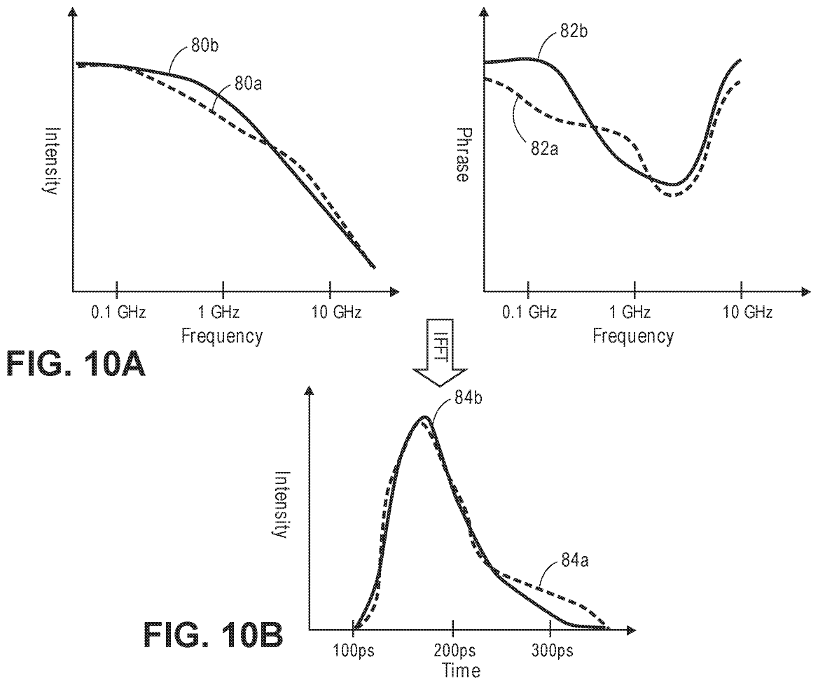

In one particular technique, the processor 26 analyzes the detected signal light 42 in the frequency domain at one or more frequencies to determine the occurrence of the fast-optical signal along the optical path 14 (see FIG. 10A), and analyzes the signal light 42 in the time domain at one or more optical path lengths to determine the spatial depth of the fast-optical signal along the optical path 14 (see FIG. 10B).

The occurrence of a fast-optical signal in the brain 12 along the optical path 14 may be determined in response to changes in the intensity profile information 80a and/or phase profile information 82a within or across multiple frequencies. For example, referring first to FIG. 10A, the processor 26 is configured for determining the occurrence of the fast-optical signal in the brain 12 along the optical path 14 by comparing a difference between the current intensity profile information 80a and current phase profile information 82a of the detected signal light 42 to baseline signal light. In the illustrated embodiment, the baseline signal light is a user-specific model in the form of baseline intensity profile information 80b and baseline phase profile information 82b derived from previously detected signal light. The user-specific model can, e.g., be derived from previous intensity profile information and phase profile information acquired from signal light detected during a previous measurement period or measurement periods.

The current intensity profile information 80a and current phase profile information 82a of the detected signal light 42 can be respectively compared to the baseline intensity profile information 80b and baseline phase profile information 82b at a relevant frequency or frequencies within the frequency domain.

For example, the greatest difference between the current intensity profile information 80a and the baseline intensity profile information 80b occurs around 1 GHz, and the greatest difference between the current phase profile information 82a and the baseline phase profile information 82b occur around 0.2 GHz, as illustrated in FIG. 10A. In this case, the differences between the current intensity profile information 80a and the baseline profile information 80b can be analyzed at frequencies near 1 GHz, and the difference between the current phase profile information 82a and the baseline profile information 82b can be analyzed at frequencies near 0.2 GHz. Knowledge of these relevant frequencies can be known prior to the current measurement period, or can otherwise be determined during the current measurement period based on a comparison between the current intensity profile information 80a and current phase profile information 82a and the baseline intensity profile information 80b and baseline phase profile information 82b.

In any event, a relatively large difference between the intensity profile information 80a and the baseline intensity profile information 80b at 1 GHz, and a relatively large difference between the phase profile information 82a and the baseline phase profile information 82b at 0.2 GHz, tend to indicate the presence of the fast-optical signal along the optical path 14, whereas a relatively small difference between the intensity profile information 80a and the baseline intensity profile information 80b at 1 GHz, and a relatively small difference between the phase profile information 82a and the baseline phase profile information 82b at 0.2 GHz, tend to indicate the absence of the fast-optical signal along the optical path 14.

Alternatively, rather than focusing on a specific frequency or specific set of frequencies in the frequency domain, the processor 26 make perform a curve fitting technique across the entire frequency range that results in single correlation values (e.g., or other metrics indicating agreement of curve fit, such as, e.g., mean squared error relative to the baseline hypothesis, inferred likelihood of data given the baseline hypothesis, etc.) respectively indicating the extent to which the current intensity profile information 80a and current phase profile information 82a respectively correlate to the baseline intensity profile information 80b and baseline phase profile information 82b. Depending on the correlation function used, a relatively small correlation coefficient value between the current intensity profile information 80a and the baseline intensity profile information 80b, and a relatively small correlation coefficient value between the current phase profile information 82a and the baseline phase profile information 82b, tend to indicate the presence (or absence) of the fast-optical signal along the optical path 14, whereas a relatively large correlation coefficient value between the current intensity profile information 80a and the baseline intensity profile information 80b, and a relatively large correlation coefficient value between the current phase profile information 82a and the baseline phase profile information 82b, tend to indicate the absence (or presence) of the fast-optical signal along the optical path 14.

Although both of the intensity profile information 80a and the phase profile information 82a has been described as being equally used by the processor 26 to determine the occurrence of a fast-optical signal along the optical path 14, it should be appreciated that consideration of the intensity profile information 80a and the phase profile information 82a acquired during the current measurement period can be weighted by the processor 26 in determining the occurrence of a fast-optical signal along the optical path 14, or one of the intensity profile information 80a and the phase profile information 82a acquired during the current measurement period can be completely ignored by the processor 26 all together in determining the occurrence of a fast-optical signal along the optical path 14.

In an optional embodiment, differences between the current measurement (i.e., the current intensity profile information 80a and current phase profile information 82a) and the baseline measurement (i.e., the baseline intensity profile information 80b and baseline phase profile information 82b) other than the differences correlated to the occurrence of a fast-optical signal within the brain 12 along the optical path 14 can be eliminated or minimized by gating the current and baseline measurements to other signals, such as an electroencephalography (EEG), patient behavior, patient stimulus (auditory, visual, sensor, situational), etc. In this manner, key frequency bands for neural coding can be identified.

With reference to FIG. 10B, the processor 26 is configured for analyzing the signal light 42 in the time domain at one or more optical path lengths to determine the spatial depth of the fast-optical signal along the optical path 14. The processor 26 accomplishes this by first transforming the frequency domain representation of the current intensity profile information 80a and current phase profile information 82a of the signal light 42 into a time domain representation (e.g., by using an Inverse Fast Fourier Transform (IFFT)) to obtain current intensity-optical path length information 84a (i.e., time-of-flight (TOF) profile information) of the detected signal light 42. The processor 26 is configured for determining the spatial depth of the fast-optical signal in the brain 12 by comparing a difference between the current TOF profile information 84a to the TOF profile expected of baseline signal light. In the illustrated embodiment, the baseline signal light is a user-specific model in the form of baseline TOF profile information 84b derived from previously detected signal light. The user-specific model can, e.g., be derived from previous TOF profile information transformed from the frequency domain representation of the signal light (i.e., the baseline intensity profile information 80b and the baseline phase profile information 82b) detected during a previous measurement period or measurement periods.

The current TOF profile information 84a can be respectively compared to the baseline TOF profile information 84b at an optical path length or path lengths within the time domain.

For example, the greatest difference between the current TOF profile information 84a and the baseline TOF profile information 84b occurs around 250 ps, as illustrated in FIG. 10B. Because TOF information can be correlated to spatial depth information (i.e., the tail end of the TOF profile information contains relatively deep information, whereas the front end of the TOF profile information contains relatively shallow information), the processor 26 may derive the spatial depth of the fast-optical signal along the optical path 14. That is, it is known that the occurrence of the fast-optical signal along the optical path 14 will perturb the sample light 40 at the depth of the fast-optical signal along the optical path 14, thereby changing the intensity of the portion of the sample light 40 having an optical path length corresponding to that depth. In this case, the intensity of the sample light 40 has substantially increased at 250 ps, and therefore, it can be assumed that the fast-optical signal occurs in the brain 12 at the depth corresponding to the optical path length of 250 ps.

In alternative embodiments, the processor 26 analyzes the detected signal light 42 in the frequency domain at one or more frequencies to determine both the occurrence of the fast-optical signal along the optical path 14 and the spatial depth of the fast-optical signal along the optical path 14; analyzes the detected signal light 42 in the time domain at one or more optical path lengths to determine both the occurrence of the fast-optical signal along the optical path 14 and the spatial depth of the fast-optical signal along the optical path 14; or analyzes the detected signal light 42 in the time domain at one or more optical path lengths to determine the occurrence of the fast-optical signal along the optical path 14, and the detected signal light 42 in the frequency domain at one or more frequencies to determine the spatial depth of the fast-optical signal along the optical path 14.

It is also possible to input the intensity and phase data from each source-detector pair into a computer simulation embodying a solver for the frequency-dependent diffusion equation, which solver contains a set of parameters reflective of optical properties at different depths or tissue locations, and then attempt to invert this equation to recover a spatial map of absorption and/or path-length changes across the brain, for example by iteratively adjusting parameters to maximize the likelihood of the intensity and phase data given the simulated solution and a model of the system noise or adjusting such parameters in the manner of gradient descent optimization or other optimization procedures. Compared to prior art, performing this inversion with a large set of frequencies that extend into the multi-GHz regime can improve the spatial resolution of the reconstructed map as well as its sensitivity to changes due to fast optical signals.

Although the processor 26 has been described as analyzing the detected signal light separately to determine the occurrence and spatial depth of the fast-optical signal based on the intensity profile information 80a, phase profile information 82a, and/or TOF profile information 84a, and geometrically deriving the two-dimensional spatial information from the multiple optical paths 14, the processor 26 may alternatively be configured for recovering three-dimensional spatial information from the intensity profile information 80a, phase profile information 82a, and/or TOF profile information 84a using diffused optical tomography (DOT)-based inverse solvers, as described in T. Durduran, et al., “Diffuse Optics for Tissue Monitoring and Tomography,” Rep. Prog. Phys., Vol. 73. No. 7, Jun. 2, 2010), or decorrelation (DCS)-based inverse solvers, as described in D. Boas, et al., “Scattering and Imaging with Diffuse Temporal Field Correlations,” Physical Review Letter, Vol. 75, No. 9, pp. 1855-1858, September 1995. For example, the processor 26 may (1) acquire the intensity profile information 80a and phase profile information 80b for each optical path 14, transform the intensity profile information 80a and phase profile information 80b from the frequency domain representation to the time domain representation to obtain the TOF profile information 84a for each optical path 14; (2) apply an inverse solver to the TOF profile information 84a for all optical paths 14 from one or more detectors to obtain a complex measure of absorption and phase shift, which may be averaged over the optical paths 14; and (3) compare the complex measure of absorption and phase shift to previously acquired measures of absorption and phase shift to look for changes that are indicative of fast-optical signals.

In one example of an inverse solver technique, the intensity profile information 80a and phase profile information 82a at each of the frequencies for each of the optical paths 14 is acquired to generate a measurement sequence A_j(f), where A is the measurement, j is the jth optical path 14 (i.e., the jth source-detector pair), and f is the frequency. The technique then computes an IFFT of the set of measurements A_j(f) to create time-of-flight (TOF) profiles B_j(t) for t=1 to N time bins that discretize the TOF profile for each optical path. The technique also generates TOF models or simulations of light passing through the head, C_j(t), by inputting differentially spatially varying patterns of absorption (μₐ) and scattering (μₛ) coefficients, and then attempts to match the detected TOF profile B_j(t) with the varying patterns of absorption (μₐ(x,y,z)) and scattering (μₛ(x,y,z)) coefficients of the TOF models or simulations C_j(t). The goal is to select the best spatially varying pattern of absorption (μₐ) and scattering (M coefficients that results in a modeled or simulated set of TOF profiles, C_j(t), that match as close as possible to the detected TOF profiles B_j(t), e.g., by solving a minimization problem that can take the following form:

minimize ∥B_j(t)−f(μₐ(x,y,z), μₛ(x,y,z))∥+R(f), with respect to μₐ(x,y,z) and μₛ(x,y,z). Here, f is the model or simulation that can generate example modeled or simulated values of C_j(t), ∥ ∥ represents some equation that compares B and f to create a measure of error (e.g., a norm), and R is some regularization function, for example one that makes sure that the distribution of absorption and scattering coefficient values inside the tissue is physically plausible (e.g., smooth). Such minimization may be carried out via gradient descent optimization, Newton's method, grid search, random search, or other optimization techniques.

A time-domain model to which detected TOF profiles can be compared to as described in the techniques above, may be an intensity impulse response function of a biological tissue parameterized by a known parametric function or model, such as a multi-exponential decay characterized by multiple biological time constants, that can be determined as a function of time and of an optical path 14, i.e., as a function of spatial location. Alternatively, a more complex parametric model can be used with distinct parameters as a function of tissue depth or biological makeup, e.g., superficial cortex versus deep cortex versus cerebrospinal fluid, skull and skin, and possibly depending on a geometric model of the subject's head or other body part under examination.

A frequency domain model to which detected intensity and phase profile information can be compared to as described in the techniques above, can be transformed from the time-domain model. In particular, to extract the values of the tissue-state-dependent model parameters, the time-domain model may be analytically subjected to a Fourier transform (FFT) to obtain a function of modulation frequency. This function may be multiplied in the frequency domain with the FFT of the time-domain light source modulation (which will often take the form of a Dirac Delta Function at 0 frequency, corresponding to a DC component, combined with a set of delta functions at the modulation frequencies), for a given modulation frequency, and subsequently subjected analytically to an IFFT to determine an expected response at that modulation frequency. This may be repeated for all of the different modulation frequencies used, to obtain an expected response as a function of modulation frequency (i.e., the frequency domain model). This expected response will be parameterized by the tissue parameters that is determined as a function of space and time.

Model fitting, such as nonlinear least squares or other standard function optimization techniques, may then be applied to fit the expected response model to the observed tissue response as a function of modulation frequency (i.e., in the frequency domain), e.g., its intensity and/or phase components separately as determined by the lock-in amplifiers. This results in extraction of estimates of the tissue-state-dependent model parameters, e.g., in the case of a multi-exponential decay, the multiple time constants, and in the case of a depth-dependent model, the tissue parameters such as absorption and scattering properties (e.g., scattering length and anisotropy factor) as a function of tissue depth, type and location. These biological parameters may be used as highly depth-specific real-time signals for a brain computer interfacing application; in addition, by distinguishing absorption from scattering properties, they may be highly specific to the neural signals of origin, e.g., neural versus hemodynamic signals. In addition, post-processing may be applied to these signals, e.g., temporal and spatial filtering based on known models of hemodynamic, neural, motion and other responses, and/or models of predicted neural, hemodynamic and motion responses, or others, in order to extract from these time-varying estimated tissue parameters a set of signals specific to neural, hemodynamic or motion based variables.

Although the controller 24 and processor 26 are described herein as being separate components, it should be appreciated that portions or all functionality of the controller 24 and processor 26 may be performed by a single computing device. Furthermore, although all of the functionality of the controller 24 is described herein as being performed by a single device, and likewise all of the functionality of the processor 26 is described herein as being performed by a single device, such functionality each of the controller 24 and the processor 26 may be distributed amongst several computing devices. Moreover, it should be appreciated that those skill in the art are familiar with the terms “controller” and “processor,” and that they may be implemented in software, firmware, hardware, or any suitable combination thereof.

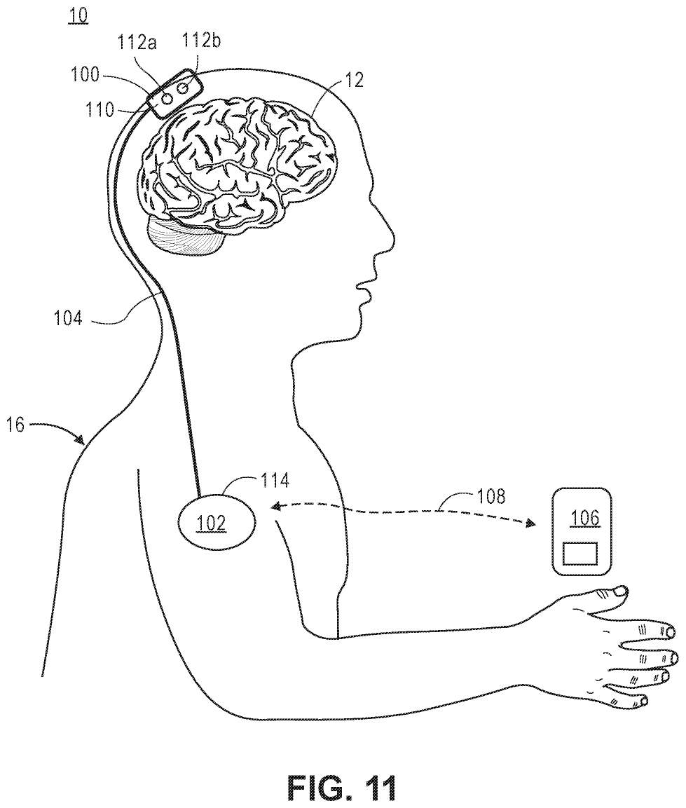

Referring now to FIG. 11, the physical implementation of the optical non-invasive measurement system 10 for use in detecting and localizing a fast-optical signal in the brain 12 of a user 16 will be described. As shown, the optical non-invasive measurement system 10 includes a wearable unit 100 that is configured for being applied to the user 16, and in this case, worn on the head of the user 16; an auxiliary head-worn or non-head-worn unit 102 (e.g., worn on the neck, shoulders, chest, or arm) coupled to the wearable unit 100 via a wired connection 104 (e.g., electrical wires); and an optional remote processor 106 in communication with the patient-wearable auxiliary unit 102 coupled via a wired connection 108 (e.g., electrical wires). Alternatively, the optical non-invasive measurement system 10 may use a non-wired connection (e.g., wireless radio frequency (RF) signals (e.g., Bluetooth, Wifi, cellular, etc.) or optical links (e.g., fiber optic or infrared (IR)) for providing power to or communicating between the respective wearable unit 100 and the auxiliary unit 102, and/or a wired connection between the auxiliary unit 102 and the remote processor 106.

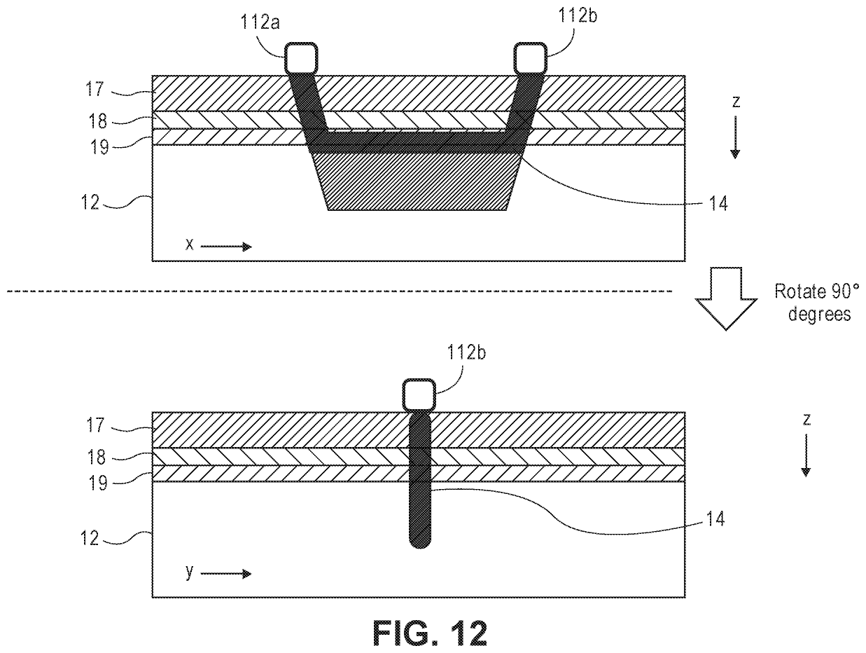

In the illustrated embodiment, the wearable unit 100 includes a support structure 110 that either contains or carries at least a portion of the optical source assembly 20 (including the optical source 64), at least a portion of the optical detection assembly 22 (including the optical detector 66) (shown in FIG. 3). The support structure 110 may take the form of a circuit board for carrying the componentry. The circuit board may be stiff or flexible, flat or curved. The wearable unit 100 may also include an output port 112a from which the sample light 40 generated by the optical source assembly 20 is emitted from the optical source 64, and an input port 112b into which the signal light 42 is input into the optical detector 66. It should be appreciated that although the input port 112b is illustrated in close proximity to the input port 112a, the proximity between the input port 112b and the output port 112a may be any suitable distance. The support structure 110 may be shaped, e.g., have a banana, headband, cap, helmet, beanie, other hat shape, or other shape adjustable and conformable to the user's head, such that the ports 112a and 112b are in close contact with the outer skin of the body part, and in this case, the scalp of the user 16, as better illustrated in FIG. 12. An index matching fluid may be used to reduce reflection of the light generated by the optical source assembly 20 from the outer skin of the scalp. An adhesive or belt (not shown) can be used to secure the support structure 110 to the brain 12 of the user 16.

The auxiliary unit 102 includes a housing 114 that contains the controller 24 and the processor 26 (shown in FIG. 1). In some embodiments, portions of the controller 24 and processor 26 may be integrated within the wearable unit 100. The auxiliary unit 102 may additionally include a power supply (which if head-worn, may take the form of a rechargeable or non-chargeable battery), a control panel with input/output functions, a display, and memory. Alternatively, power may be provided to the auxiliary unit 102 wirelessly (e.g., by induction). The auxiliary unit 102 may further include the other portions of the optical source assembly 20 (including the signal generator 60 and amplifier 62) and other portions of the optical detection assembly 22 (including the amplifier 68 and ADC 70). The remote processor 106 may store image data from previous sessions, and include a display screen.

As shown in FIG. 12, the ports 112a, 112b may be placed against the scalp 17 of the user 16, such that sample light 40 first passes through the scalp 17, skull 18, and cerebral spinal fluid (CSF) 19 along a relatively straight path, enter the brain tissue 12, then exit in reverse fashion along a relatively straight path through the CSF 19, skull 18, and scalp 17, thereby creating a banana-shaped optical path 14. As depicted in the top half of FIG. 12, the greater distance of the optical path 14 may be across the x-y plane as compared to its distance along the z-direction.

Thus, the optical path 14 will be defined by the location of the output port 112a (which is associated with the optical source 64) and the location of the input port 112b (which is associated with the optical detectors 66). In the case of a single fixed source-detector arrangement, only one optical path 14 can be created with the optical measurement system 10. However, as discussed above, the optical measurement system 10 may be modified, such that it can sequentially or simultaneously detect physiological events in multiple spatially resolved optical paths 14. Multiple optical paths 14 can be created either by making the output port 112a and input port 112b movable relative to each other and/or spacing multiple output ports 112a and/or input ports 112b relative each other.

For example, in the case of a single source-detector arrangement, as shown in the optical non-invasive measurement system 10 of FIG. 3, a single movable output port 112a may be moved around at different locations 116a-116h across the scalp 17 along a predetermined path 116, as shown in FIG. 13. At each location 116a-116h along the predetermined path 116, the light emitted by the output port 112a enters and exits the brain 14 (see FIG. 12) into input port 112b. In effect, this creates a multitude of optical paths 14 (or geometric paths) through the brain tissue 14 that are imaged while the output port 112a moves along the path 116 over multiple measurement periods. Although the predetermined path 116 in FIG. 13 is circular, the predetermined path 116 can follow any geometry, including rectangular, triangular, etc. The fields of view of the input port 112b with respect to the output port 112a at the various locations along the predetermined path 116 may have areas of overlap and/or may have little or no overlap.

The multiple optical paths 14 may facilitate the generation of a high-resolution functional map of the upper layer of cortex of the brain 12 with spatial resolution given by the x-y plane (i.e., along the plane of the scalp 17) confinement of the paths, in the manner of tomographic volume reconstruction. Moreover, moving the output port 112a with respect to the input port 112b at one or more pre-determined locations may probe a region of interest from multiple angles and directions. That is, the output port 112a will be create multiple optical paths 14 extending from the pre-determined location of the path 116 to the multiple input ports 112b, allowing optical data from the pre-determined location at the origin of each of multiple optical paths 14 to be acquired along multiple axes. Optical data taken across multiple axes across a region of interest may facilitate the generation of a 3-D map of the region of interest. Optical data received by the input ports 112b may be used to generate images with comparable resolution in the z-direction (i.e., perpendicular to a scalp 17 as in the x-y plane (i.e., along the scalp 17), and/or may allow optical probing or interrogation of larger region in brain tissue 12 (e.g., across multiple optical paths 14 over a surface of the scalp 17).

As another example, in the case of a multiple source-multiple detector arrangement, as shown in the optical measurement system 10′ of FIG. 5, the multiple output ports 112a and multiple input ports 112b may be tiled across the scalp 17, as illustrated in FIG. 14. In this case, each optical path 14 (geometric path) is defined by a given output port 112a (which is associated with the optical source 64) at a given location and a given input port 112b (which is associated with the optical detectors 66) at a given location. Thus, the output ports 112a and input ports 112b are located at fixed positions on the scalp 17. In effect, this creates a multitude of optical paths 14 (or geometric paths) through the brain 12 within a single measurement period.

The output ports 112a and input ports 112b may be arranged in any desirable pattern over the scalp 17. In the illustrated embodiment, four output ports 112a are provided for the four optical sources 64 (four on the sides), and five input ports 112b are provided for the four optical detectors 66 (four on the corners and one in the center). However, the output ports 112a and input ports 112b may be arranged or located in a symmetric or asymmetric array and/or may be arranged in a circular or radial pattern or a rectangular-shaped pattern. The fields of view of the output ports 112a and input ports 112b with respect to each other may have areas of overlap and/or may have little or no overlap. In some variations, the output ports 112a or input ports 112b may be tiled adjacent to each other, such that the individual fields-of-view are adjacent to each other with little or no overlap.

In the same manner described above with respect to FIG. 13, the multiple optical paths 14 may facilitate the generation of a high-resolution functional map of the upper layer of cortex of the brain 12 with spatial resolution given by the x-y plane (i.e., along the plane of the scalp 17) confinement of the paths, and furthermore, allowing optical data from the pre-determined location at the origin of each of multiple optical paths 14 to be acquired along multiple axes.

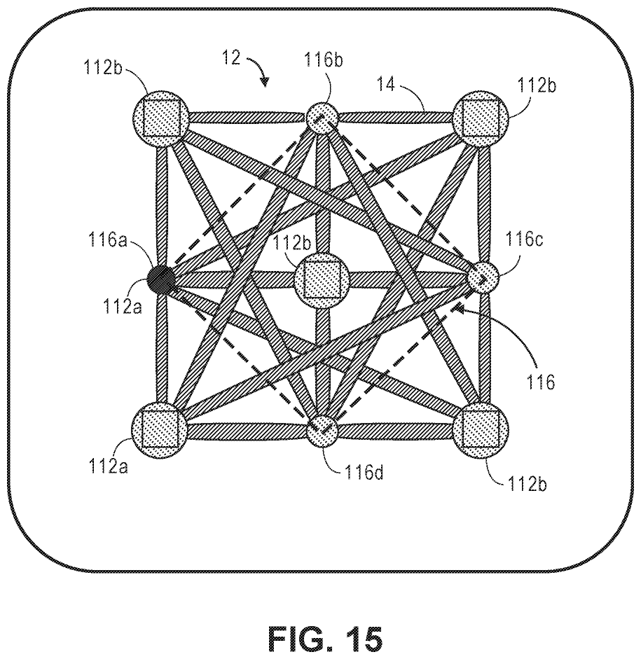

As still another example, in the case of a single-source multi-detector arrangement, as shown in the optical measurement system 10″ of FIG. 7, multiple input ports 112b may be tiled across the scalp 17, as illustrated in FIG. 15. The input ports 112b may be arranged in any desirable pattern over the scalp 17. In the illustrated embodiment, five input ports 112b are provided for the five optical detectors 66 (four on the respective sides between the corners). However, the input ports 112b may be arranged or located in a symmetric or asymmetric array and/or may be arranged in a circular or radial pattern or a rectangular-shaped pattern. The fields of view of the input ports 112b with respect to output port 112a have areas of overlap and/or may have little or no overlap. In some variations, the input ports 112b may be tiled adjacent to each other, such that the individual fields-of-view are adjacent to each other with little or no overlap.

The single output port 112a may be fixed relative to the input ports 112b, in effect, creating a multitude of optical paths 14 (or geometric paths) through the brain 12 within a single measurement period. However, in the illustrated embodiment, the output port 112a is moved around at different locations 116a-116d across the scalp 17 along a predetermined path 116, thereby creating additional optical paths 14 over a multitude of measurement periods. Although the predetermined path 116 in FIG. 15 is diamond-shaped, the predetermined path 116 can follow any geometry, including rectangular, triangular, circular, etc.

At each location along the predetermined path 116, the light emitted by the output port 112a enters and exits the brain 12 (see FIG. 12) into the multiple input ports 112b. In effect, this creates a multitude of optical paths 14 (or geometric paths) through the brain 12 under the scalp 17 that are imaged while the output port 112a moves along the path 116.

In the same manner described above with respect to FIG. 13, the multiple optical paths 14 may facilitate the generation of a high-resolution functional map of the upper layer of cortex of the brain 12 with spatial resolution given by the x-y plane (i.e., along the plane of the scalp 17) confinement of the geographic paths, and furthermore, allowing optical data from the pre-determined location at the origin of each of multiple optical paths 14 to be created along multiple axes.

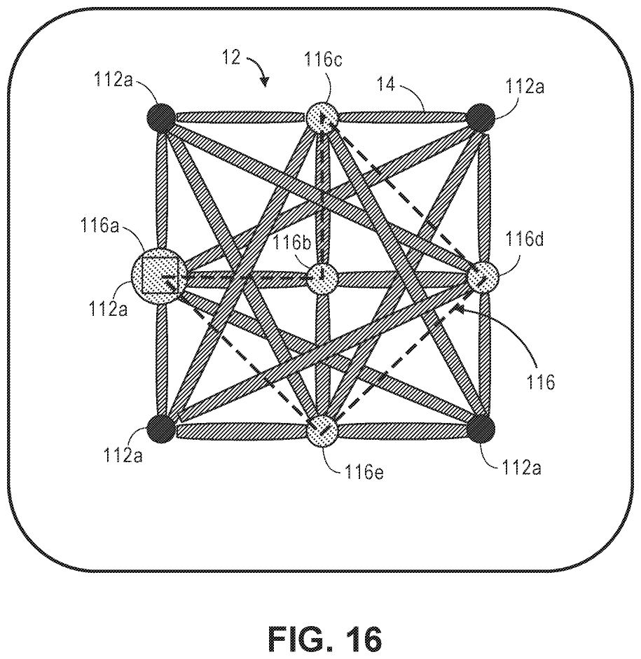

As yet another example, in the case of a multi-source single-detector arrangement, as shown in the optical measurement system 10′″ of FIG. 9, multiple output ports 112a may be tiled across the scalp 17, as illustrated in FIG. 16. The output ports 112a may be arranged in any desirable pattern over the scalp 17. In the illustrated embodiment, four output ports 112a are provided for the four optical sources 64 (four on the respective sides between the corners). However, the output ports 112a may be arranged or located in a symmetric or asymmetric array and/or may be arranged in a circular or radial pattern. The fields of view of the output ports 112a with respect to input port 112b have areas of overlap and/or may have little or no overlap. In some variations, the output ports 112a may be tiled adjacent to each other, such that the individual fields-of-view are adjacent to each other with little or no overlap.

The single input port 112b may be fixed relative to the output ports 112a, in effect, creating a multitude of optical paths 14 (or geometric paths) through the brain 12 within a single measurement period. However, in the illustrated embodiment, the input port 112b is moved around at different locations 116a-116e across the scalp 17 along a predetermined path 116, thereby creating additional optical paths 14 over a multitude of measurement periods. Although the predetermined path 116 in FIG. 15 is irregularly-shaped, the predetermined path 116 can follow any geometry. At each location of the input port 112b along the predetermined path 116, the light emitted by the output ports 112a enters and exits the brain 12 (see FIG. 12) into the input port 112b. In effect, this creates a multitude of optical paths 14 (or geometric paths) through the brain 12 under the scalp 17 that are imaged while the input port 112b moves along the path 116.

In the same manner described above with respect to FIG. 13, the multiple optical paths 14 may facilitate the generation of a high-resolution functional map of the upper layer of cortex of the brain 12 with spatial resolution given by the x-y plane (i.e., along the plane of the scalp 17) confinement of the geographic paths, and furthermore, allowing optical data from the pre-determined location at the origin of each of multiple optical paths 14 to be acquired along multiple axes.

Although the optical non-invasive measurement systems 10 have been described herein as having a one-to-one correspondence between the optical sources and the output ports 112a, with the output ports 112a being capable of being moved relative to the input ports 112b to create additional optical paths 14, it should be appreciated that multiple fixed output ports 112a may be associated with a single optical source to create additional optical paths 14. For example, to mimic a moving optical source, the sample light 40 output by the optical source may be sequentially scanned to the output ports 112a over multiple measurement periods using galvanic mirrors, or the output ports 112a may take the form of multiple static optical fibers fixed between the scalp 17 and the optical source, and an optical switch can direct the sample light 40 from the optical source to the optical fibers over multiple measurement periods.

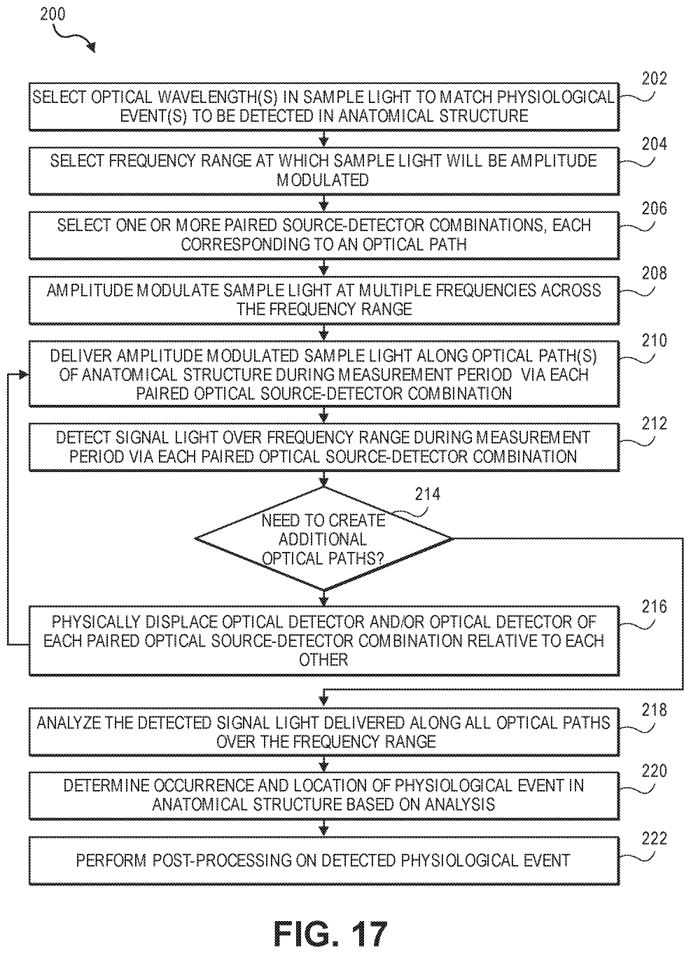

Referring to FIG. 17, having described the structure and function of the optical measurement system 10 (and the variations thereof), one particular method 200 performed by the optical measurement system 10 to non-invasively image the brain 12 will now be described.

First, the optical wavelength(s) of the sample light 42 is selected to match the physiological event(s) to be detected in the brain 12 (step 202). In this case, the physiological event is a fast-optical signal, in which case, one optical wavelength may be greater than 850 nm. In the case where it is desirable to additionally detect blood oxygen concentration, another optical wavelength may be selected to be in the range of 650 nm to 750 nm.

Next, the frequency range at which the sample light 40 will be intensity modulated is selected (e.g., 100 MHz to 10 GHz) (step 204). One or more paired optical source-detector combinations, each corresponding to an optical path 14, are then defined (step 206). The paired optical source-detector combination(s) may be defined using a single optical source and a single optical detector (e.g., the single-source single-detector arrangement of the optical measurement system 10 of FIGS. 1 and 3), such that a single optical path 14 is defined between the single optical source and the single optical detector; multiple optical sources and multiple optical detectors (e.g., the multi-source multi-detector arrangement of the optical measurement system 10′ of FIGS. 4-5), such that different optical paths 14 are defined between each respective optical source and each respective optical detector; a single optical source and multiple optical detectors (e.g., the single-source multi-detector arrangement of the optical measurement system 10″ of FIGS. 6-7), such that a different optical path 14 is defined between the single optical source and each respective optical detector; or multiple optical sources and a single optical detector (e.g., the multi-source single-detector arrangement of the optical measurement system 10″ of FIGS. 8-9), such that a different optical path 14 is defined between each respective optical source and the single optical detector.

Next, sample light 40 is intensity modulated at multiple frequencies across the frequency range via each of the paired optical source-detector combination(s) (step 208). The sample light 40 may be sequentially intensity modulated at the multiple frequencies, e.g., by sweeping a frequency of the intensity modulated sample light over the frequency range within the measurement period, or the sample light 40 may be simultaneously intensity modulated at the multiple frequencies.

Next, via each paired optical source-detector combination, the intensity modulated sample light 40 is delivered along the optical path(s) 14 in the brain 12 during a single measurement period, such that the intensity modulated sample light 40 is scattered by the brain 12, resulting in signal light 42 that exits the brain 12 (step 210). In the case where a single source and a single detector is used to define a single paired source-detector combination, the intensity modulated sample light 40 will be delivered along a single optical path 14 of the brain 12 during the measurement period (see FIG. 13). In the case where multiple sources and/or multiple detectors are used to define multiple paired source-detector combinations, the intensity modulated sample light 40 will be delivered along multiple optical paths 14 in the brain 12 during the measurement period (see FIGS. 14-16).

Next, via each paired optical source-detector combination, the signal light 42 is detected over the frequency range within the measurement period (step 212). Then, if additional optical paths 14 need to be created (step 214), the optical source (i.e., the output port) and/or optical detector (i.e., the input port) of each paired optical source-detector combination are physically displaced relative to each other (step 216). With respect to the multi-detector arrangement of the optical measurement system 10′ of FIGS. 4-5, no physical displacement between the optical sources and optical detectors may necessary, although it can also be used to further increase the effective number of source-detector pairs. With respect to the single-source single-detector arrangement of the optical measurement system 10 of FIGS. 1 and 3, the single-source multi-detector arrangement of the optical measurement system 10″ of FIGS. 6-7, and the multi-source single-detector arrangement of the optical measurement system 10″ of FIGS. 8-9, the single optical source and/or single optical detector of these arrangements may be physically displaced.

The process then returns to step 208 where sample light 40 is intensity modulated at multiple frequencies within the frequency via each of the paired optical source-detector combination(s) (step 208), the intensity modulated sample light 40 is delivered along the additional optical path(s) 14 of the brain 12 during the next measurement period (step 210), and the signal light 42 is detected over the frequency range within the next measurement period via each paired optical source-detector combination (step 212). Then, if necessary (step 214), the optical source and optical detector of each paired optical source-detector combination are physically displaced relative to each other (step 216).

If additional optical paths 14 need not be created (step 214) (i.e., all the necessary optical paths 14 have been created), the detected signal light 42 is analyzed for all optical paths 14 over the respective frequency range (step 218), and an occurrence and a location of a physiological event (in this case, a fast-optical signal) in at least two dimensions within the brain 12 is determined based on the analysis (step 220).

Post-processing can then be performed on the determined fast-optical signal and any other detected physiological events (step 222), and in the case where the anatomical structure 12 comprises brain matter, such post-processing may comprise determining the level of neural activity within the brain 12 based on the determined occurrence and location of the fast-optical signal in the brain 12.

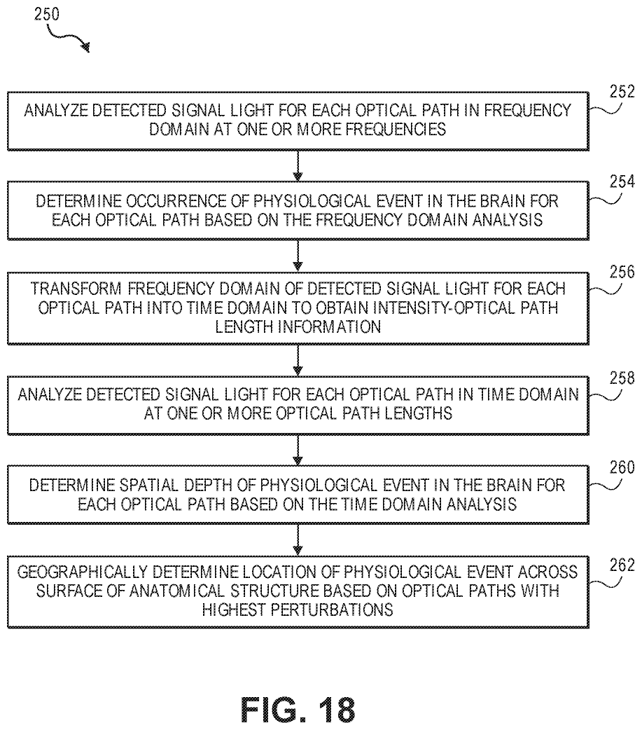

In a preferred method 250 illustrated in FIG. 18, the occurrence and the location of the fast-optical signal within the brain 12 is determined in three dimensions, including the spatial depth within the brain 12. In particular, the detected signal light 42 for each optical path 14 is analyzed in the frequency domain at one or more frequencies (e.g., by comparing a difference between the detected signal light 42 to a baseline signal light (e.g., a user-specific model)) (step 252), and the occurrence of the fast-optical signal in the brain 12 is determined based on this analysis (step 254). Next, the frequency domain representation of the detected signal light 42 for each optical path 14 is transformed into the time domain representation (e.g., using an IFFT) to obtain intensity-optical path length information (i.e., a profile of the expected dispersion of an optical pulse over time, which offers a direct measure of the intensity and phase of light across each path length) the detected signal light 42 (step 256), the detected signal light 42 for each optical path 14 is analyzed in the time domain at one or more optical path lengths (e.g., by comparing a difference between the detected signal light 42 to a baseline signal light (e.g., a user-specific model)) (step 258), and the spatial depth of the fast-optical signal in the brain 12 is determined based on this analysis (step 260).

In alternative embodiments, the occurrence of the fast-optical signal in the brain 12 can be determined based on an analysis of the detected signal light 42 in the time domain and/or the spatial depth of the fast-optical signal in the brain 12 can be determined based on an analysis of the detected signal light 42 in the frequency domain.

Regardless of the manner in which the occurrence and spatial depth of the fast-optical signal in the brain 12 are determined, the location of the fast-optical signal is determined in the x-y plane by geographically determining the location of the fast-optical signal based on tissue point-spread functions 14 with highest perturbation (e.g., by determining the highest differences between the respective detected signal light 42 and the baseline signal light (step 262).

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. An optical non-invasive measurement system, comprising:
an optical source assembly configured for intensity modulating sample light at multiple frequencies within a frequency range, and delivering the intensity modulated sample light along one or more optical paths in an anatomical structure during a single measurement period, such that the intensity modulated sample light is scattered by the anatomical structure, resulting in signal light that exits the anatomical structure;
an optical detection assembly configured for detecting the signal light over the frequency range within the measurement period; and
a processor configured for analyzing the detected signal light, and, based on this analysis, determining an occurrence and spatial depth of a physiological event in the anatomical structure.

2. The optical non-invasive measurement system of claim 1, wherein the processor is configured for analyzing the detected signal light in the frequency domain at one or more frequencies, and based on this analysis, determining the occurrence of the physiological event in the anatomical structure.

3. The optical non-invasive measurement system of claim 2, wherein the processor is configured for determining the occurrence of the physiological event in the anatomical structure by comparing a difference between the detected signal light to a baseline signal light at the one or more frequencies.

4. The optical non-invasive measurement system of claim 3, wherein the baseline signal light comprises a user-specific model.

5. The optical non-invasive measurement system of claim 1, wherein the processor is configured for analyzing the detected signal light in the time domain at one or more optical path lengths, and based on this analysis, determining the occurrence of the physiological event in the anatomical structure.

6. The optical non-invasive measurement system of claim 5, wherein the processor is configured for transforming a frequency domain representation of the detected signal light into a time domain representation of the detected signal light to obtain a measure of the detected signal light as a function of optical path length, wherein the occurrence of the physiological event in the anatomical structure is determined based on the measure of the detected signal light as a function of optical path length.

7. The optical non-invasive measurement system of claim 6, wherein the processor is configured for computationally transforming the frequency domain representation of the detected signal light into the time domain representation of the signal light using an Inverse Fast Fourier Transform (IFFT).

8. The optical non-invasive measurement system of claim 5, wherein the processor is configured for determining the occurrence of the physiological event in the anatomical structure by comparing a difference between the detected signal light to baseline signal light at the one or more optical path lengths.

9. The optical non-invasive measurement system of claim 8, wherein the baseline signal light comprises a user-specific model.

10. The optical non-invasive measurement system of claim 1, wherein the processor is configured for analyzing the detected signal light in the time domain at one or more optical path lengths, and based on this analysis, determining the spatial depth of the physiological event in the anatomical structure.

11. The optical non-invasive measurement system of claim 10, wherein the processor is configured for transforming a frequency domain representation of the detected signal light into a time domain representation of the detected signal light to obtain a measure of the detected signal light as a function of optical path length, wherein the spatial depth of the physiological event in the anatomical structure is determined based on the measure of the detected signal light as a function of optical path length.

12. The optical non-invasive measurement system of claim 11, wherein the processor is configured for computationally transforming the frequency domain representation of the detected signal light into the time domain representation of the detected signal light using an Inverse Fast Fourier Transform (IFFT).

13. The optical non-invasive measurement system of claim 10, wherein the processor is configured for determining the spatial depth of the physiological event in the anatomical structure by comparing a difference between the detected signal light to baseline signal light at the one or more optical path lengths.

14. The optical non-invasive measurement system of claim 13, wherein the baseline signal light comprises a user-specific model.

15. The optical non-invasive measurement system of claim 1, wherein the processor is configured for analyzing the detected signal light in the frequency domain at one or more frequencies, and based on this analysis, determining the spatial depth of the physiological event in the anatomical structure.

16. The optical non-invasive measurement system of claim 15, wherein the processor is configured for determining the spatial depth of the physiological event in the anatomical structure by comparing a difference between the detected signal light to baseline signal light at the one or more frequencies.

17. The optical non-invasive measurement system of claim 16, wherein the baseline signal light comprises a user-specific model.

18. The optical non-invasive measurement system of claim 1, wherein the anatomical structure is a brain, and the physiological event is an occurrence of a fast-optical signal.

19. The optical non-invasive measurement system of claim 18, wherein the sample light has a wavelength equal to or greater than 850 nm.

20. The optical non-invasive measurement system of claim 1, wherein the sample light has a wavelength in the range of 350 nm to 1800 nm.

21. The optical non-invasive measurement system of claim 1, further comprising a controller configured for instructing the optical source assembly to sequentially intensity modulate sample light at the multiple frequencies over the frequency range within the measurement period.

22. The optical non-invasive measurement system of claim 21, wherein the controller is configured for instructing the optical source assembly to sequentially intensity modulate sample light at the multiple frequencies by sweeping the intensity modulation frequency of the intensity modulated sample light over the frequency range within the measurement period.

23. The optical non-invasive measurement system of claim 21, further comprising a controller configured for instructing the optical source assembly to simultaneously intensity modulate sample light at the multiple frequencies.

24. The optical non-invasive measurement system of claim 1, wherein the sample light has two different optical wavelengths, and the processor is configured for analyzing the detected signal light, and, based on this analysis, determining an occurrence and spatial depth of the physiological event in the anatomical structure at the first optical wavelength, and determining an occurrence and spatial depth of another physiological event in the anatomical structure at the second optical wavelength, the physiological event and other physiological event being of different types.

25. The optical non-invasive measurement system of claim 24, wherein the first optical wavelength has a wavelength equal to or greater than 850 nm, the second optical wavelength is in the range of 650 nm to 750 nm, the physiological event is a fast-optical signal, and the other physiological event is a change in blood oxygen concentration.

26. The optical non-invasive measurement system of claim 1, wherein the frequency range comprises a frequency equal to or greater than 2 GHz.

27. The optical non-invasive measurement system of claim 1, wherein the frequency range comprises a frequency equal to or greater than 5 GHz.

28. The optical non-invasive measurement system of claim 1, wherein the frequency range comprises 1 GHz to 5 GHz.

29. The optical non-invasive measurement system of claim 1, wherein the frequency range comprises 100 MHz to 10 GHz.

30. The optical non-invasive measurement system of claim 1, wherein the optical source assembly comprises:
an electrical signal generator configured for outputting an electrical alternating current (AC) signal at the multiple frequencies;
a first amplifier configured for amplifying the AC signal and outputting a drive signal; and
an optical source configured for outputting the intensity modulated sample light at the multiple frequencies in accordance with the drive signal.

31. The optical non-invasive measurement system of claim 30, wherein the optical source comprises one of a vertical-cavity surface-emitting laser (VCSEL), a light emitting diode (LED), an edge emitting diode laser, and a flash lamp.

32. The optical non-invasive measurement system of claim 1, wherein the optical detection assembly comprises:
an optical detector configured for detecting the signal light and outputting an electrical physiological-encoded signal;
a second amplifier configured for amplifying the physiological-encoded signal; and
an analog-to-digital converter (ADC) configured for digitizing the amplified physiological-encoded signal into digital physiological-encoded data.

33. The optical non-invasive measurement system of claim 32, wherein the second amplifier is a lock-in amplifier configured for, in response to an electrical signal output by the optical source assembly at the multiple frequencies, amplifying the physiological-encoded signal comprises outputting an intensity and phase of the physiological-encoded signal, wherein the ADC is configured for digitizing the intensity and phase output by the lock-in amplifier into digital physiological-encoded data.

34. The optical non-invasive measurement system of claim 32, wherein the optical detector comprises at least one discrete detector.

35. The optical non-invasive measurement system of claim 34, wherein the at least one discrete detector comprises a single discrete detector.

36. The optical non-invasive measurement system of claim 34, wherein each of the at least one discrete detector has an area greater than 30 μm².

37. The optical non-invasive measurement system of claim 34, wherein each of the at least one discrete detector has an area greater than 200 μm².

38. The optical non-invasive measurement system of claim 34, wherein each of the at least one discrete detector has an area less than 1000 μm².

39. The optical non-invasive measurement system of claim 32, wherein the optical detector comprises a photodiode.

40. An optical non-invasive measurement method, comprising:
intensity modulating sample light at multiple frequencies within a frequency range;
delivering the intensity modulated sample light along an optical path in an anatomical structure during a single measurement period, such that the intensity modulated sample light is scattered by the anatomical structure, resulting in signal light that exits the anatomical structure;
detecting the signal light over the frequency range within the measurement period;
analyzing the detected signal light;
determining an occurrence and spatial depth of a physiological event in the anatomical structure based on the analysis.

41. The optical non-invasive measurement method of claim 40, wherein the detected signal light is analyzed in the frequency domain at one or more frequencies, and the occurrence of the physiological event in the anatomical structure is based on the analysis in the frequency domain.

42. The optical non-invasive measurement method of claim 41, wherein the occurrence of the physiological event in the anatomical structure is determined by comparing a difference between the detected signal light to a baseline signal light at the one or more frequencies.

43. The optical non-invasive measurement method of claim 42, wherein the baseline signal light comprises a user-specific model.

44. The optical non-invasive measurement method of claim 40, wherein the detected signal light is analyzed in the time domain at one or more optical path lengths, and the occurrence of the physiological event in the anatomical structure is determined based on the analysis in the time domain.

45. The optical non-invasive measurement method of claim 44, further comprising transforming a frequency domain representation of the detected signal light into a time domain representation to obtain intensity-optical path length information of the detected signal light, wherein the occurrence of the physiological event in the anatomical structure is determined based on intensity-optical path length information.

46. The optical non-invasive measurement method of claim 45, wherein the frequency domain representation of the detected signal light is transformed into the time domain representation of the detected signal light using an Inverse Fast Fourier Transform (IFFT).

47. The optical non-invasive measurement method of claim 44, wherein the occurrence of the physiological event in the anatomical structure is determined by comparing a difference between the detected signal light to baseline signal light at the one or more optical path lengths.

48. The optical non-invasive measurement method of claim 47, wherein the baseline signal light comprises a user-specific model.

49. The optical non-invasive measurement method of claim 40, wherein the detected signal light is analyzed in the time domain at one or more optical path lengths, and the spatial depth of the physiological event in the anatomical structure is determined based on the analysis in the time domain.

50. The optical non-invasive measurement method of claim 49, further comprising transforming a frequency domain representation of the detected signal light into the time domain representation of the detected signal light to obtain intensity-optical path length information of the detected signal light, wherein the spatial depth of the physiological event in the anatomical structure is determined based on intensity-optical path length information.

51. The optical non-invasive measurement method of claim 50, wherein the frequency domain representation of the detected signal light is transformed into the time domain of the detected signal light using an Inverse Fast Fourier Transform (IFFT).

52. The optical non-invasive measurement method of claim 49, wherein the occurrence of the physiological event in the anatomical structure is determined by comparing a difference between the detected signal light to baseline signal light at the one or more optical path lengths.

53. The optical non-invasive measurement method of claim 52, wherein the baseline signal light comprises a user-specific model.

54. The optical non-invasive measurement method of claim 40, wherein the detected signal light is analyzed in the frequency domain at one or more frequencies, and the spatial depth of the physiological event in the anatomical structure is determined based on the analysis in the frequency domain.

55. The optical non-invasive measurement method of claim 54, wherein the spatial depth of the physiological event in the anatomical structure is determined by comparing a difference between the detected signal light to baseline signal light at the one or more frequencies.

56. The optical non-invasive measurement method of claim 55, wherein the baseline signal light comprises a user-specific model.

57. The optical non-invasive measurement method of claim 40, wherein the anatomical structure is a brain, and the physiological event is an occurrence of a fast-optical signal.

58. The optical non-invasive measurement method of claim 57, wherein the sample light has a wavelength equal to or greater than 850 nm.

59. The optical non-invasive measurement method of claim 40, wherein the sample light has a wavelength in the range of 350 nm to 1800 nm.

60. The optical non-invasive measurement method of claim 40, wherein the sample light is sequentially intensity modulated at the multiple frequencies.

61. The optical non-invasive measurement method of claim 60, wherein the sample light is sequentially intensity modulated at the multiple frequencies by sweeping the intensity modulation frequency of the intensity modulated sample light over the frequency range within the measurement period.

62. The optical non-invasive measurement method of claim 40, wherein the sample light is simultaneously intensity modulated at the multiple frequencies.

63. The optical non-invasive measurement method of claim 40, wherein the sample light has two different optical wavelengths, and the processor is configured for analyzing the detected signal light, and, based on this analysis, determining an occurrence and spatial depth of the physiological event in the anatomical structure at the first optical wavelength, and determining an occurrence and spatial depth of another physiological event in the anatomical structure at the second optical wavelength, the physiological event and other physiological event being of different types.

64. The optical non-invasive measurement method of claim 63, wherein the first optical wavelength has a wavelength equal to or greater than 850 nm, the second optical wavelength is in the range of 650 nm to 750 nm, the physiological event is a fast-optical signal, and the other physiological event is a change in blood oxygen concentration.

65. The optical non-invasive measurement method of claim 40, wherein the frequency range comprises a frequency equal to or greater than 2 GHz.

66. The optical non-invasive measurement method of claim 40, wherein the frequency range comprises a frequency equal to or greater than 5 GHz.

67. The optical non-invasive measurement method of claim 40, wherein the frequency range comprises 1 GHz to 5 GHz.

68. The optical non-invasive measurement method of claim 40, wherein the frequency range comprises 100 MHz to 10 GHz.

69. The optical non-invasive measurement method of claim 40, wherein intensity modulating the sample light at multiple frequencies within a frequency range comprising outputting an electrical alternating current (AC) signal at the multiple frequencies, amplifying the AC signal and outputting a drive signal, and outputting the intensity modulated sample light at the multiple frequencies in accordance with the drive signal.

70. The optical non-invasive measurement method of claim 69, wherein the intensity modulated sample light is generating by one of a vertical-cavity surface-emitting laser (VCSEL), a light emitting diode (LED), an edge emitting diode laser, and a flash lamp.

71. The optical non-invasive measurement method of claim 40, wherein detecting the intensity modulated signal light comprises detecting the signal light and outputting an electrical physiological-encoded signal, amplifying the physiological-encoded signal, and digitizing the amplified physiological-encoded signal into digital physiological-encoded data.

72. The optical non-invasive measurement method of claim 71, wherein amplifying the physiological-encoded signal comprises outputting an intensity and phase of the physiological-encoded signal in response to an electrical signal output at the multiple frequencies, wherein the intensity and phase is digitized into digital physiological-encoded data.

73. The optical non-invasive measurement method of claim 72, wherein the intensity modulated signal light is detected with at least one discrete detector.

74. The optical non-invasive measurement method of claim 73, wherein the at least one discrete detector comprises a single discrete detector.

75. The optical non-invasive measurement method of claim 73, wherein each of the at least one discrete detector has an area greater than 30 μm².

76. The optical non-invasive measurement method of claim 73, wherein each of the at least one discrete detector has an area greater than 200 μm².

77. The optical non-invasive measurement method of claim 73, wherein each of the at least one discrete detector has an area less than 1000 μm².

78. The optical non-invasive measurement method of claim 73, wherein the optical detector comprises a photodiode.

79.-161. (canceled)
